(19) United States
(12) Patent Application Publication
(10) Pub. No.: US 2024/0206516 A1
(43) Pub. Date: Jun. 27, 2024

(54) SYSTEMS AND METHODS FOR SUGAR-REDUCTION AND/OR FIBER PRODUCTION FOR FOOD AND OTHER APPLICATIONS

(71) Applicants: President and Fellowa of Harvard College, Cambridge, MA (US); Massachusetts Institute of Technology, Cambridge, MA (US)

(72) Inventors: Donald E. Ingber, Cambridge, MA (US); Vaskar Gnyawali, Cambridge, MA (US); Daneille Harrier, Cambridge, MA (US); Yan Liu, Cambridge, MA (US); Evan Minghao Zhao, Cambridge, MA (US); David A. Weitz, Cambridge, MA (US); James J. Collins, Newton, MA (US); Adama Marie Sesay, Cambridge, MA (US); Bobby Tyrell Haney, Cambridge, MA (US)

(73) Assignees: President and Fellows of Harvard College, Cambridge, MA (US); Massachusetts Institute of Technology, Cambridge, MA (US)

(21) Appl. No.: 18/568,085

(22) PCT Filed: Jun. 8, 2022
(86) PCT No.: PCT/US2022/032613

§ 371 Date: Dec. 7, 2023

Related U.S. Application Data
(60) Provisional application No. 63/208,473, filed on Jun. 8, 2021.

(51) Int. Cl. A23L 33/20; A23L 29/00; A23L 29/30; A23L 33/125; A23L 33/135; C12N 11/12

(52) U.S. Cl. CPC A23L 33/20; A23L 29/06; A23L 29/30; A23L 33/125; A23L 33/135; C12N 11/12; C12Y 204/01009

(57) ABSTRACT

The present disclosure generally relates to sugar reduction in foods and, in some aspects, to enzyme-polymer conjugated particles for food and other applications. Certain aspects of the disclosure are directed to compositions for reducing sugar content and/or producing dietary fiber within food products during or after consumption (e.g., in a subject's gastrointestinal (GI) tract), while maintaining the sweetness and flavor of the sugar in food products upon consumption (e.g., in a subject's mouth). For example, in one set of embodiments, a composition may comprise a particle comprising an enzyme capable of converting a sugar into a relatively non-digestible form (e.g., a polymer), optionally an inhibitor that reversibly inhibits the enzyme from converting the sugar, and optionally an additive capable of associating with the inhibitor. The composition may be used for in situ conversion of sugars upon exposure to an environment condition (e.g., pH and/or temperature) in the GI tract.

### RELATED APPLICATIONS

This application claims the benefit of U.S. Provisional Patent Application Ser. No. 63/208,473, filed Jun. 8, 2021, entitled “Systems and Methods for Sugar-Reduction for Food and Other Applications,” incorporated herein by reference in its entirety.

### FIELD

The present disclosure generally relates to sugar reduction in foods and, in some aspects, to enzyme-polymer conjugated particles for food and other applications.

### BACKGROUND

Sugar is an important food ingredient in food products. Worldwide consumption of sugar has increased due to consumer lifestyles and choices. Recent studies have shown that high consumption of sugar can have negative effects on one's health and may lead to chronic health conditions and diseases such as obesity, diabetes, cardiovascular diseases, dementia, and tooth decay.

Many methods of reducing sugar in food products are available, including replacing sugar with substitutes (e.g., artificial sweeteners) that contain significantly less calories compared to sugar. However, such methods may compromise the authentic flavor (e.g., sweetness) of the food products, and thus more effective methods and compositions for sugar reduction in food products are still needed.

### SUMMARY

The present disclosure generally relates to sugar reduction in foods and, in some aspects, to enzyme-polymer conjugated particles for food and other applications. The subject matter of the present disclosure involves, in some cases, interrelated products, alternative solutions to a particular problem, and/or a plurality of different uses of one or more systems and/or articles.

In one aspect, the present disclosure is generally directed to a composition. According to one set of embodiments, the composition comprises a particle comprising an enzyme capable of polymerizing a sugar to produce a polymer; and an inhibitor associated with the enzyme, wherein the inhibitor inhibits the enzyme from polymerizing the sugar, and wherein the inhibitor is dissociated from the enzyme when the particle is exposed to an ionic strength of at least 5 mmol/L and a pH of greater than 3.5.

In some embodiments, the composition comprises a particle comprising an enzyme capable of polymerizing a monosaccharide or a disaccharide to produce a polysaccharide; and an inhibitor associated with the enzyme, wherein the inhibitor inhibits the enzyme from polymerizing the monosaccharide or disaccharide.

In some embodiments, the composition comprises a particle comprising one or more enzymes, wherein the one or more enzymes comprises an enzyme selected from the group consisting of glucose oxidase, glucose dehydrogenase, glycosyltransferase, fructosyltransferase, hexosyltransferase, sucrase, amylosucrase, glucansucrase, levansucrase, inulosucrase, fructose dehydrogenase, glucose-fructose oxidoreductase, beta-galactosidase, amylase, cellulase, and laccase; and a polyphenol associated with the one or more enzymes.

In some embodiments, the composition comprises digestive juice comprising a particle, wherein the particle comprises an enzyme capable of polymerizing a sugar to produce a polymer; and an inhibitor capable of inhibiting the enzyme from polymerizing the sugar.

In some embodiments, the composition comprises a particle comprising an enzyme capable of converting a sugar from an original form into a form that is non-digestible or less digestible compared to the original form; and an inhibitor that inhibits the enzyme from converting the sugar, wherein upon a change in a condition associated with the composition, the enzyme converts the sugar into the non-digestible or less digestible form, and wherein the condition comprises pH, temperature, and/or ionic strength.

In some embodiments, the composition comprises a particle, comprising one or more enzymes, wherein the one or more enzymes comprises an enzyme selected from the group consisting of glucose oxidase, glucose dehydrogenase, glycosyltransferase, fructosyltransferase, hexosyltransferase, sucrase, amylosucrase, glucansucrase, levansucrase, inulosucrase, fructose dehydrogenase, glucose-fructose oxidoreductase, beta-galactosidase, amylase, cellulase, laccase; and an inhibitor that inhibits the enzyme from converting the sugar.

In some embodiments, the composition comprises a particle comprising an enzyme capable of polymerizing a sugar to produce a polymer.

In some embodiments, the composition comprises a particle comprising one or more enzymes, wherein the one or more enzymes comprises an enzyme selected from the group consisting of glucose oxidase, glucose dehydrogenase, glycosyltransferase, hexosyltransferase, fructosyltransferase, sucrase, amylosucrase, glucansucrase, levansucrase, inulosucrase, fructose dehydrogenase, glucose-fructose oxidoreductase, beta-galactosidase, amylase, cellulase, laccase.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 is a schematic illustration showing a composition comprising a particle that comprises an enzyme, an inhibitor, and an additive, in accordance with some embodiments;

FIG. 2 is a schematic illustration showing the composition (same as FIG. 1) upon exposure to an environmental condition; in accordance with some embodiments;

FIG. 3 is a schematic illustration showing the composition (same as FIG. 2) interacting with a sugar, in accordance with some embodiments;

FIG. 4 is a schematic illustration showing the experimental design of a composition comprising an enzyme for sugar reduction, in accordance with some embodiments;

FIG. 5 is graph of the enzymatic activity of glucose oxidase in the presence of tannic acid and Mg⁺² at different pH conditions, in accordance with some embodiments;

FIG. 6 is a graph of the enzymatic activity of glucose oxidase in the presence of EGCE (Epigallocatechin gallate) and Mg⁺² at different pH conditions, in accordance with some embodiments;

FIGS. 7A-7B are micrographs of a microgel containing a shell of agarose cross-linked with pectin surrounding a fluidic droplet, in accordance with some embodiments;

FIG. 8A is a graph of fructose concentration in the presence of fructose dehydrogenase-containing alginate-polyethylene diacrylate particles, in accordance with some embodiments;

FIG. 8B is a graph of the fructose concentration in the presence of fructose dehydrogenase-containing alginate particles, in accordance with some embodiments;

FIG. 9 is a schematic illustration showing the attachment of a conjugated enzyme-carboxymethyl cellulose complex to nanocellulose, in accordance with some embodiments;

FIG. 10A is a graph of percent (%) sucrose reduction in the presence of inulosucrase, in accordance with some embodiments;

FIG. 10B is a graph of Inulin (mg/ml) produced from reduced sugar in the presence of inulosucrase, in accordance with some embodiments;

FIG. 10C is a graph of percent (%) sugar composition of food (e.g. sucrose solution) in the presence of inulosucrase, in accordance with some embodiments;

FIG. 10D is a graph of percent (%) sucrose reduction in the presence of inulosucrase-conjugated nanocellulose, in accordance with some embodiments;

FIG. 10E is a graph of percent (%) sugar composition of food (e.g. sucrose solution) in the presence of inulosucrase-conjugated nanocellulose, in accordance with some embodiments;

FIG. 10F is a graph of inulin (mg/ml) produced from reduced sugar in the presence of free inulosucrase in different digestive buffers, in accordance with some embodiments;

FIG. 10G is a graph of inulin (mg/ml) produced from reduced sugar in the presence of inulosucrase-conjugated nanocellulose in different digestive buffers, in accordance with some embodiments;

FIG. 10H is a spectrum of MALDI-TOF characterizing the presence of inulin, in accordance with some embodiments;

FIG. 11A is a micrograph of a microfluidic device used to produce pectin based microparticles; in accordance with some embodiments;

FIG. 11B is a micrograph of pectin based microparticles, in accordance with some embodiments;

FIG. 12A is a graph illustrating storage stability of pectin particles at pH of 5.5, in accordance with some embodiments;

FIGS. 12B-12C are a set of micrographs of pectin based microparticles exposed to a citric acid buffer for 1 day (FIG. 12B) and for 8 days (FIG. 12C), in accordance with some embodiments;

FIG. 12D is a graph illustrating storage stability of pectin particles at pH of 6.5, in accordance with some embodiments;

FIG. 13A is a graph of particle size of pectin based microparticles in different pH buffers that simulate various gastric fluids, in accordance with some embodiments;

FIG. 13B is a graph of percent (%) of sucrose change in the presence of free inulosucrase and inulosucrase-containing pectin particles, in accordance with some embodiments;

FIG. 14A is a micrograph of an ES100 microcapsule, in accordance with some embodiments;

FIG. 14B is a series of micrographs of ES100 microcapsules stored in different pH buffers, in accordance with some embodiments;

FIG. 15A is a schematic illustration showing a microparticle having an alginate core and an ES100 shell, in accordance with some embodiments;

FIG. 15B is series of micrographs of the microparticles (left) in FIG. 15A upon exposure to a pH 7.5 buffer (middle) and after 1 minute in the pH 7.5 buffer (right), in accordance with some embodiments;

FIG. 16A is a schematic illustration showing a microparticle having an alginate core, an ES100 inner shell, and an alginate outer shell, in accordance with some embodiments; and

FIG. 16B is series of micrographs of the microparticles (left) from FIG. 16A upon exposure to a pH 7.5 buffer (middle) and after 1 minute in the pH 7.5 buffer (right), in accordance with some embodiments;

FIGS. 17A-17B are micrographs of microgels having a pectin shell containing a fluid droplet containing nanocellulose-conjugated inulosucrase, in accordance with some embodiments;

FIG. 18A is a schematic illustration of a microgel having a shell comprising agarose crosslinked with pectin, in accordance with some embodiments;

FIG. 18B is a schematic illustration of a microgel having two shells comprising an inner pectin shell and an outer agarose shell, in accordance with some embodiments;

FIG. 19 is a graph illustrating the effect of tannic acid on enzymatic activity, in accordance with some embodiments;

FIGS. 20A-20C are graphs illustrating the effect of adding inulosucrase to Capri San® (FIG. 20A), unsweetened chocolate (FIG. 20B), and sweetened chocolate (FIG. 20C), in accordance with some embodiments;

FIG. 21 is a schematic illustration of the formation of polymeric particles encapsulating nanocellulose-enzyme complex with polyphenol, in accordance with some embodiments;

FIGS. 22A-22D are representative images of cells on the apical channel of primary human small intestine chips, in accordance with some embodiments;

FIGS. 22E-22F are graphs illustrating transepithelial barrier assessment assay of primary cells on the intestine chip, before and after 24 hours treatment using nanocellulose (FIG. 23E) and nanocellulose-enzyme conjugate (FIG. 23F), in accordance with some embodiments;

FIG. 22G is a graph illustrating Alamar blue assay of primary cells on the intestine chips, in accordance with some embodiments; and

FIGS. 23A-23B are graphs illustrating the effect of tannic acid-enzyme (TA-Enz) complexes on sucrose reduction in sucrose solution and apple juice, in accordance with some embodiments.

### DETAILED DESCRIPTION

The present disclosure generally relates to sugar reduction in foods and, in some aspects, to enzyme-polymer conjugated particles for food and other applications. Certain aspects of the disclosure are directed to compositions for reducing sugar content and/or producing dietary fibers within food products during or after consumption (e.g., in a subject's gastrointestinal (GI) tract), while maintaining the sweetness and flavor of the sugar in food products upon consumption (e.g., in a subject's mouth) and before being absorbed by the body. For example, in one set of embodiments, a composition may comprise a particle comprising an enzyme capable of converting a sugar into a relatively non-digestible form (e.g., a polymer such as a prebiotic fiber), optionally an inhibitor that reversibly inhibits the enzyme from converting the sugar, and optionally an additive capable of associating with the inhibitor. In some embodiments, a composition may comprise a particle that comprises an enzyme but lacks an inhibitor and/or an additive described herein. The composition may be used for in situ conversion of sugars upon exposure to an environmental condition (e.g., pH and/or temperature) in the GI tract. In addition, some aspects are directed to methods for making or using such compositions, kits associated with such compositions, or the like.

Sugar is an important food ingredient in food products and is the generic name given to carbohydrates that impart food products with the sweet taste. Carbohydrates can be sorted into three subtypes: monosaccharides, disaccharides, and polysaccharides. Soluble simple carbohydrates commonly present in food products include glucose, fructose, sucrose, and lactose. Recent studies have shown that high consumption of sugar may lead to chronic health conditions and diseases. A major challenge for food product producers is therefore trying to provide healthier options by reducing the amount of sugar without compromising the authentic taste (e.g., sweetness) of the food products. Accordingly, certain aspects of the disclosure are directed to compositions that can be used to reduce the sugar content in food products after consumption, while maintaining the authentic flavor (e.g., sweetness) of the food product upon or after consumption.

In one example, the composition may include a particular beneficial combination of ingredients that allows for a stimuli-triggered (e.g., pH and/or temperature) enzymatic conversion of the sugar to a non-metabolizable form in the GI tract. For instance, the composition may include an enzyme capable of converting (e.g., degrading, polymerizing, etc.) the sugar, optionally an inhibitor capable of reversibly deactivating the enzyme, and optionally one or more additives capable of interacting with the inhibitor and/or the enzyme. Advantageously, such a composition may remain stable in the presence of a sugar prior to consumption, e.g., such that authentic flavor of the food product is maintained, while allowing for stimuli-triggered reduction of sugar content after consumption. In some cases, the sugar, after consumption, may be converted into a non-digestible fiber having certain health benefits. Methods of making and using compositions such as these are also described in more detail below.

For example, in some cases, the enzyme and/or the inhibitor may be contained within particles. The particles may be consumed by a subject to affect the amount of sugar absorbed by the subject. In one case, the particles may be pre-mixed with a beverage and the enzymes within the particles may remain inactive in the beverage until after being ingested. Once ingested and delivered to the GI tract, the particles may become activated such that the enzymes can facilitate the reduction of available sugar within the beverage. In another instance, the particles may be mixed within the food eaten by the subject, for example, during preparation of the food, immediately before consumption (e.g., similar to a condiment), and/or taken separately by the subject (for example, before or after ingesting the food). In some cases, the particle may comprise a polymer (e.g., a polymer shell or body), for instance, that comprises a polymer that can be ingested by the subject. Non-limiting examples of such polymers include cellulose, glucomannan, polyvinyl alcohol, pectin, alginate, agarose, gelatin, inulin, amylose, phytogel, nanocellulose fiber, xanthan gum, or others such as those described herein. It should be noted that other types of particles (e.g., protein-based and/or phospholipid-based particles) may also be used.

In some embodiments, the composition comprises a particle comprising one or more enzymes. The one or more enzymes may comprises an enzyme selected from glycosyltransferases (including but not limited to fructosyltransferase, hexosyltransferase, sucrase, amylosucrase, glucansucrase, levansucrase, inulosucrase), oxidoreductases (including but not limited to glucose oxidase, fructose dehydrogenase, glucose-fructose oxidoreductase), dehydrogenases (including but not limited to glucose dehydrogenase, fructose dehydrogenase), and hydrolases (including but not exclusive to beta-galactosidase, amylase, cellulase, laccase).

In some embodiments, a composition is generally directed to a particle comprising an enzyme and an inhibitor associated with the enzyme. FIG. 1 shows an illustration of such an embodiment. As shown, a composition comprises a particle 10 that includes an enzyme 16 and an inhibitor 18 associated with the enzyme, for example, non-covalently bound to the enzyme. In some embodiments, the enzyme, e.g., when not bound to the inhibitor, is capable of converting a sugar (e.g., a sugar in a food product) from an original form into a form that is non-digestible (e.g., non-metabolizable) or less digestible (e.g., less metabolizable) compared to the original form. In some embodiments, a sugar of non-digestible or less digestible form is a dietary fiber. For example, as described in more detail below, the enzyme may be an enzyme that is capable of degrading, transforming, or polymerizing the sugar from a more metabolizable form into a less metabolizable or a non-metabolizable form during or after consumption.

In one set of embodiments, the composition comprises a particle comprising an enzyme capable of polymerizing a sugar (e.g., a sugar in the food product) to produce a polymer. The sugar may be any of a variety of monosaccharides or disaccharides, or other sugars. The polymer that is produced may be any of a variety of polysaccharides. For example, the composition may comprise a particle comprising an enzyme capable of polymerizing a monosaccharide or a disaccharide to produce a polysaccharide, e.g., one that is non-digestible or at least less digestible. Non-limiting examples of monosaccharides and disaccharides include glucose, sucrose, fructose, lactose, galactose, amylopectin, starch, and maltose. Non-limiting examples of polysaccharides that such sugars can be polymerized to form include oligosaccharides (e.g., amylose), fructo-oligosaccharides, malto-oligosaccharides, reuteran, gluco-oligosaccharides, branched oligo-fructose: levan, inulin.

In some embodiments, the polymer that is produced from the sugar is a prebiotic. For example, fructooligosaccharides (e.g., inulin, levan, etc.) are prebiotics that act as dietary fibers that can be used by certain gut bacteria to promote gut health.

In some embodiments, the enzyme capable of polymerizing a sugar (e.g., in the food product) to produce a polymer may be a glycosyltransferase. Glycosyltransferases are enzymes that establish natural glycosidic linkages between monosaccharides. In some embodiments, glycosyltransferases (e.g., fructo-oligosaccharides, etc.) may facilitate the formation of beta glycosidic linkages (e.g., linkages that resists hydrolysis by digestive enzymes due to a lack of enzymatic recognition) between monosaccharides. Non-limiting examples of glycosyltransferase include fructosyltransferase. Specific non-limiting examples include sucrose:fructose fructosyltransferase, fructose:fructose fructosyltransferase, hexosyltransferase, sucrase, amylosucrase, glucansucrase, levansucrase, inulosucrase. In some embodiment, the composition may comprise one or more of the enzymes described above. For example, in one set of embodiments, the composition may comprise a combination of amylosucrase and inulosucrase, and/or other combinations of enzymes.

In one set of embodiments, the composition comprises a particle comprising an enzyme capable of degrading or transforming a sugar (e.g., a sugar in the food product) from a form that can be readily metabolized (or catalyzed) into a form that cannot be as readily metabolized by the GI tract. In some embodiments, the sugar may be a monosaccharide or a disaccharide (e.g., glucose, sucrose, fructose, etc.). The enzymes that may be used to degrade or transform such sugars include glucose oxidase, fructose dehydrogenase, and glucose-fructose oxidoreductase. For example, glucose oxidase is an oxidoreductase that is associated with the oxidation of a sugar (e.g., glucose) into a different form (e.g., gluconic acid) that is not readily metabolized (or catalyzed). Specific functions for enzymes such as these are described in more detail later.

In some embodiments, the composition comprises a particle comprising an enzyme that is capable of breaking glycosidic bonds in a sugar (e.g., a disaccharide, an oligosaccharide, or a polysaccharide). For example, the enzyme may be a glycosidase. Non-limiting examples of such enzymes include beta-galactosidase (lactase), amylase, cellulase, laccase. In some embodiments, the enzymes may be used to break down a sugar at a rate that is faster than the rate at which the converted sugar can be metabolized or absorbed in the GI tract, thus reducing the amount of absorbed sugar. In some embodiments, such enzymes may be used in combination with a different type of enzyme described above to facilitate the conversion and/or polymerization of a sugar to a polymerized form and/or a form that cannot be readily metabolized.

For instance, in some embodiments, the composition comprises a particle comprising a combination of enzymes described herein. In one set of embodiments, the combination may include two or more of enzymes selected from those capable of degrading or transforming a sugar (e.g., glucose oxidase), polymerizing a sugar (e.g., fructosyltransferase), and/or breaking glycosidic bonds in a sugar (e.g., lactase). Advantageously, the two or more enzymes may work synergistically to convert (e.g., catalyze) a sugar.

As mentioned above, a composition such as described herein may comprise an inhibitor associated with an enzyme, including but not limited to any of the enzymes described herein. In some embodiments, the inhibitor (e.g., inhibitor 18 in FIGS. 1-3) is capable of inhibiting the enzyme from converting the sugar from an original form into a form that is non-digestible (e.g., non-metabolizable) or less digestible (e.g., less metabolizable). For example, in one set of embodiments, the inhibitor inhibits an enzyme from polymerizing a sugar (e.g., a monosaccharide or disaccharide) to a polymer (e.g., polysaccharide) in the composition. In another set of embodiments, the inhibitor inhibits the enzyme from degrading, catalyzing or transforming the sugar in the composition from a readily metabolizable form to a form that is non-metabolizable or less metabolizable in humans. Other inhibitors may also be used in other embodiments.

In some embodiments, the inhibitor may be associated with the enzyme in the particle via any appropriate interactions, such as those described herein. The association of the enzyme with the inhibitor may render the enzyme substantially inactive within the composition, e.g., such that the enzyme cannot associate with (e.g., bind to) and convert a sugar (e.g., a sugar in a food product). For example, the inhibitor may prevent the association of the sugar to the enzyme by occupying an active site in the enzyme that is responsible for the association with the sugar, or by blocking access to the active site by a substrate, etc. In some embodiments, the enzyme may be substantially inert and stable in the presence of the sugar in the food product when associated with the inhibitor. FIG. 1 can be used to illustrate such an embodiment. As shown in this figure, inhibitor 18 may associate with enzyme 16 at an active site such that a sugar 11 is prevented from accessing the active site and bind to the enzyme.

In some embodiments, the association between the inhibitor and the enzyme may be a reversible association. For instance, the inhibitors may associate with the enzyme via any appropriate interactions, such as hydrogen bonding, Van der Waals, hydrophobic association, electrostatic, etc. The inhibitor (via competitive and/or non-competitive binding) may be, for example, any of a variety of molecules described herein. In some embodiments, the inhibitor comprises polyphenols. In some cases, the polyphenol comprises flavonoids. In one set of embodiments, as a non-limiting illustration, the inhibitor is a polyphenol selected from the group consisting of tannic acid, chlorogenic acid, quercetin, Epigallocatechi gallate (EGCG), and gallic acid.

In some embodiments, the inhibitor is capable of associating with or dissociating from the enzyme when the particle is exposed to a condition. The condition may include, e.g., pH and/or ionic strength. For example, in one set of embodiments, a binding affinity between the inhibitor and the enzyme is affected by a change in pH and/or ionic strength in the composition. The inhibitor may associate with the enzyme at a relatively low pH (e.g., less than 3.5, less than 3.3, less than 3, less than 2.5, etc.). In some cases, the inhibitor may dissociate from the enzyme at a relatively higher pH (e.g., at least 3.5, at least 4, at least 4.5, at least 5, at least 5.5, at least 6, at least 6.5, at least 6.8, etc.) and/or when exposed to a certain ionic strength (e.g., at least 5 mmol/L, at least 10 mmol/L, at least 15 mmol/L, at least 25 mmol/L, at least 30 mmol/L, at least 40 mmol/L, at least 50 mmol/L, etc.). As described in more detail herein, the composition may comprise additives (e.g., metal ions, such as those described herein) that impart the composition with a particular ionic strength.

In some embodiments, the inhibitor associates with the enzyme at relatively low pH (e.g., less than 3.5, less than 3, less than 2.5, less than 2, etc.). The ionic strength may be less than or equal to 50 mmol/L (e.g., less than or equal to 40 mmol/L, less than or equal to 30 mmol/L, less or equal to 25 mmol/L, less than or equal to 20 mmol/L, less than or equal to 15 mmol/L, less than or equal to 10 mmol/L, less than or equal to 5 mmol/L, etc.). In some embodiments, when exposed to such a relatively low pH and/or low ionic strength, at least 50% (at least 60%, at least 70%, at least 80%, at least 90%, or at least 95%, or all) of the enzymes in the particles are associated with the inhibitors.

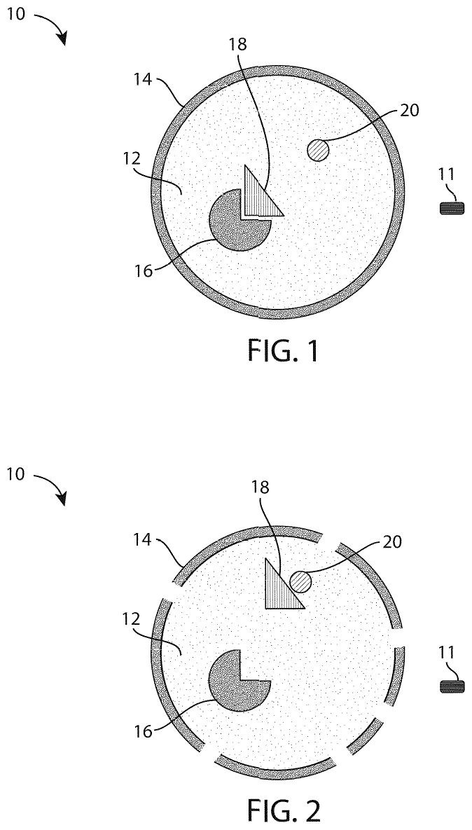

In some embodiments, the inhibitor may dissociate from the enzyme at relatively higher pH and/or when exposed to a certain ionic strength. FIG. 2 can be used to illustrate such an embodiment. As shown, inhibitor 18 may dissociate from enzyme 16 when exposed to a relatively high pH and/or ionic strength. A relatively higher pH may refer to a pH that is at least 3.5 (e.g., at least 4, at least 4.5, at least 5, at least 5.5, at least 6, at least 6.5, at least 7, at least 7.2, at least 7.4, at least 7.6, at least 8). The ionic strength may be at least 5 mmol/L (e.g., at least 10 mmol/L, at least 20 mmol/L, at least at least at least 15 mmol/L, at least 25 mmol/L, at least 30 mmol/L, at least 40 mmol/L, at least 50 mmol/L, etc.). In one set of embodiments, a substantial amount of inhibitors dissociates from the enzyme at a neutral or physiological pH. For example, when exposed to a neutral pH (e.g., pH of about 7), at least 50% (at least 60%, at least 70%, at least 80%, at least 90%, or at least 95%, or all) of the enzymes in the particle may be dissociated from the inhibitors. In one set of embodiments, the inhibitor, by associating with the enzyme, is capable of inhibiting at least 10% (e.g., at least 20%, at least 30%, at least 40%, or at least 50%, at least 60%, at least 70%, at least 80%, at least 90%, (and/or up to 95%, up to 97%, up to 99%, or up to 100%)) of the enzymatic activity at a relatively low pH (e.g., pH of about 3.5 or less). In accordance with certain embodiments, the inhibitor is capable of dissociating from the enzyme when exposed to a relatively high pH (e.g., pH of 5.5 or higher), such that the enzyme recovers at least 10% (e.g., at least 20%, at least 30%, at least 40%, or at least 50%) of the enzymatic activity.

The inhibitor may be present in any of a variety of amounts in the composition. In some embodiments, the inhibitor described herein may be present in an amount of greater than or equal to 1 micromolar, greater than or equal to 5 micromolar, greater than or equal to 10 micromolar, greater than or equal to 25 micromolar, greater than or equal to 50 micromolar, greater than or equal to 60 micromolar, greater than or equal to 70 micromolar, greater than or equal to 80 micromolar, greater than or equal to 90 micromolar, greater than or equal to 100 micromolar, greater than or equal to 150 micromolar, greater than or equal to 250 micromolar, greater than or equal to 500 micromolar, greater than or equal to 1 mM, greater than or equal to 5 mM, greater than or equal to 10 mM, greater than or equal to 25 mM, or greater than or equal to 50 mM. In some embodiments, the inhibitor described herein may be present in an amount of less than or equal to 100 mM, less than or equal to 50 mM, less than or equal to 25 mM, less than or equal to 10 mM, less than or equal to 5 mM, less than or equal to 1 mM, less than or equal to 500 micromolar, less than or equal to 250 micromolar, less than or equal to 100 micromolar, less than or equal to 90 micromolar, less than or equal to 80 micromolar, less than or equal to 70 micromolar, less than or equal to 60 micromolar, less than or equal to 50 micromolar, less than or equal to 25 micromolar, less than or equal to 10 micromolar, or less than or equal to 5 micromolar. Combination of the above-referenced range are possible (e.g., greater than or equal to 1 micromolar and less than or equal to 100 mM, or greater than or equal to 50 micromolar and less than or equal to 100 micromolar).

In some embodiments, the composition may further comprise an additive capable of associating with an inhibitor and/or an enzyme described herein. In one set of embodiments, the additive may be encapsulated within a particle. For example, referring again to FIG. 1, particle 10 may further comprise an additive 20 adjacent the enzyme 16 and the inhibitor 18. While FIG. 1 shows an embodiments in which the additive is contained within a particle, it should be understood that the disclosure is not so limited and that in certain embodiments, the additive is not contained within the particle. For example, in some cases, the composition may contain an additive that is located external of the particle. Furthermore, it should also be understood that an additive is not always required. For example, in one set of embodiments, the composition may comprise a particle comprising one or more enzymes described herein that are capable of converting a sugar (e.g., a sugar in a food product) and an inhibitor associated with the one or more enzymes, without any additive being present in the composition.

In some embodiments, the additive comprises a metal ion. For example, the additive may be a mineral comprising a metal ion. The additive, in some cases, may be capable of facilitating association or binding of the enzyme with a sugar, thereby catalyzing conversion (e.g., polymerization) of a sugar. In some embodiments, the metal ion described herein is a metal cation having an oxidation state of at least +2 or at least +3. In some cases, the metal ion is a transition metal ion and/or an alkaline earth metal ion. The term “alkaline earth metal” is used herein to refer to the following six metal elements of Group 2 of the periodic table: beryllium (Be), magnesium (Mg), calcium (Ca), strontium (Sr), barium (Ba), and radium (Ra). Non-limiting examples of alkaline earth metal ions that can be used in the composition include magnesium (e.g., Mg⁺²) and calcium (e.g., Ca⁺²). Non-limiting examples of transition metals that can be used in the composition include iron and zinc. Non-limiting examples of transition metal cations that can be used include Fe⁺³ and Zn⁺². In some embodiments, the metal may be a Group 13 metal element, e.g., aluminum. A non-limiting example of metal cations that can be used is Al⁺³. Non-limiting examples of minerals include, but are not limited to, CaCl₂, FeCl₃, FeCl₂, MgCl₂, ZnCl₂, CuCl₂, HgCl₂, HgCl, KCl, etc. Alternatively or additionally, the additive may include one or more acids, e.g., such as a ethylenediaminetetraacetic acid (EDTA).

Alternatively or additionally, the additive comprises a primer capable catalyzing enzymatic conversion of a sugar. In some embodiments, the primer comprises at least one polysaccharide such as inulin, amylose, and/or reuteran.

In an exemplary embodiment, a composition comprises a particle comprising one or more enzymes described previously that is capable of converting a sugar (e.g., a sugar in a food product), an inhibitor associated with the one or more enzymes, and an additive capable of associating with the inhibitor (e.g., as shown in FIG. 1). The additives described herein may be capable of reversibly associating with the inhibitor and/or enzyme via any of a variety of appropriate mechanisms. Non-limiting examples of such mechanisms include metal chelation, hydrophobic association, Van der Waals, electrostatic, hydrogen bonding.

In some embodiments, the additive (when present) may be capable of interacting with the enzyme and/or inhibitor to modulate the association (or dissociation) of the inhibitor with the enzyme when exposed to a certain condition (e.g., pH). For instance, FIGS. 1-3 illustrates a non-limiting embodiment of a composition in which the additive is capable of interacting with (e.g., via non-competitive or competitive association) the inhibitor to cause dissociation of the inhibitor from the enzyme. As described in more detail below, in an exemplary embodiment, the additive comprises a metal ion that may associate with an inhibitor via metal coordination.

Without wishing to be bound by any theory, in some embodiments, it is believed that a condition such as pH may be used to influence the association between an inhibitor and an enzyme and the association between an inhibitor and an additive. For example, when the particle is exposed to a first pH (e.g., a pH in a food product), a binding affinity between the inhibitor and the enzyme may be substantially greater than a binding affinity between the inhibitor and the additive. In some embodiments, a majority of the enzymes is associated with the inhibitor, such that the enzyme is inhibited from converting (e.g., polymerizing) a sugar (e.g., a sugar in the food product).

FIG. 1 can be used to illustrate such an embodiment. As shown, when particle 10 is exposed to a first pH, inhibitor 18 preferentially associates with enzyme 16 rather than additive 20. As such, enzyme 16 is incapable of binding to a sugar 11.

Without wishing to be bound by any theory, it is believed that in some embodiments, when the composition is exposed to a second pH (e.g., a pH in the GI tract), the binding affinity between the inhibitor and the enzyme may be substantially less than the binding affinity between the inhibitor and the additive. In some instances, the inhibitor may preferentially associate with the additive, thereby inhibiting the inhibitor from associating with the enzyme. As such, the enzyme may be capable of binding to and converting a sugar.

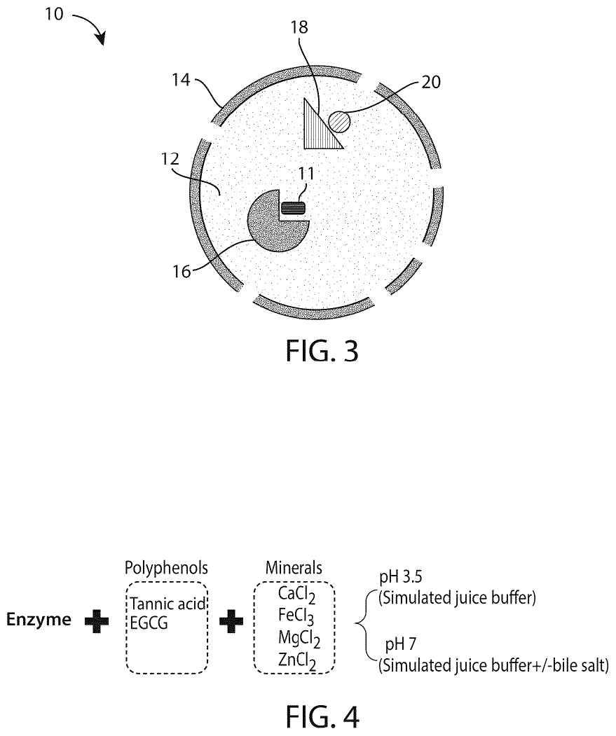

FIG. 2 can be used to illustrate such an embodiment. As shown, when particle 10 is exposed to a second pH, inhibitor 18 preferentially associates with additive 20 as opposed to enzyme 16, causing inhibitor 18 to dissociate from enzyme 16. In some cases, enzyme 16 may be available to associate with and/or convert sugar 11, e.g., as shown in FIG. 3.

In some embodiments, a binding affinity between the inhibitor and the additive may exceed a binding affinity between the inhibitor and the enzyme at a pH of at least 3.5 (e.g., at least 4, at least 4.5, at least 5, at least 5.5, at least 6, at least 6.5, at least 7, at least 7.5, or at least 8). In some embodiments, a binding affinity between the inhibitor and the enzyme may exceed a binding affinity between the inhibitor and the additive at a pH of no more than 3.5 (e.g., no more than 3, no more than 2.5, or no more than 2).

While FIGS. 1-3 shows an embodiment in which the additive is capable of interacting with the inhibitor to cause dissociation of the inhibitor from the enzyme, it should be noted that the disclosure is not so limited, and that in certain embodiments, the additive may instead interact directly with the enzyme rather than with the inhibitor. Non-limiting examples of such additives may include HgCl₂, HgCl, CuCl₂, ZnCl₂, FeCl₃, FeCl₂, EDTA, etc.

In some embodiments, the composition may comprise a particle containing one or more enzymes and an additive (e.g., a metal ion) located external the particle. In some cases, upon degradation of the particle at a relatively high pH at a location (e.g., GI tract) within a subject, the enzyme within the particle may be exposed to the additive external the particle. In some cases, the additive may interact with the enzyme in a way that advantageously promote enzymatic activity, e.g., such as facilitating binding of the enzyme with a sugar. Non-limiting examples of additives that may advantageously promote enzymatic activity include KCl, CaCl₂, MgCl₂, etc.

The additives described herein may be present in any of a variety of amounts in the composition. In some embodiments, an additive may be present in an amount greater than or equal to 1 mM, greater than or equal to 5 mM, greater than or equal to 7.5 mM, greater than or equal to 10 mM, greater than or equal to 20 mM, greater than or equal to 30 mM, greater than or equal to 40 mM, greater than or equal to 50 mM, greater than or equal to 75 mM, or greater than or equal to 100 mM in the composition. In some embodiments, an additive may be present in an amount of less than or equal to 100 mM, less than or equal to 75 mM, less than or equal to 50 mM, less than or equal to 40 mM, less than or equal to 30 mM, less than or equal to 20 mM, less than or equal to 10 mM, less than or equal to 7.5 mM, or less than or equal to 5 mM. Combination of the above-referenced range are possible (e.g., greater than or equal to 5 mM and less than or equal to 50 mM). Other range are also possible. It should be noted when the composition comprises one or more types of additives, each type of additive may individually make up or in total be present in one or more of the above-referenced ranges.

The metal ions described herein may be present in any of a variety of amounts in the composition. In some embodiments, a metal ion may be present in an amount of greater than or equal to 1 mM, greater than or equal to 5 mM, greater than or equal to 7.5 mM, greater than or equal to 10 mM, greater than or equal to 20 mM, greater than or equal to 30 mM, greater than or equal to 40 mM, greater than or equal to 50 mM, greater than or equal to 75 mM, or greater than or equal to 100 mM in the composition. In some embodiments, a metal ion may be present in an amount of less than or equal to 100 mM, less than or equal to 75 mM, less than or equal to 50 mM, less than or equal to 40 mM, less than or equal to 30 mM, less than or equal to 20 mM, less than or equal to 10 mM, less than or equal to 7.5 mM, or less than or equal to 5 mM. Combination of the above-referenced range are possible (e.g., greater than or equal to 5 mM and less than or equal to 50 mM). Other range are also possible. It should be noted when the composition comprises one or more types of metal ions, each type of metal ions may individually make up or in total be present in one or more of the above-referenced ranges.

In an exemplary embodiment, a composition comprises a particle comprising one or more enzymes described herein that is capable of converting a sugar (e.g., a sugar in a food product), a polyphenol associated with the one or more enzymes, and optionally an additive comprising a metal ion. Without wishing to be bound by any theory, it is hypothesized that the metal ion may be capable of associating with polyphenol via metal coordination. The enzyme, additive, metal ion, and polyphenol may be any of a variety of enzymes, additives, and metal ions described previously. In another embodiment, the composition may comprise a particle comprising one or more enzymes described herein and an additive (e.g., a metal ion), without any polyphenol present within the particle.

Without wishing to be bound by any theory, it is believed that the association between the polyphenol and the one or more enzymes and the association between the polyphenol and the metal ion (when present) may be pH-dependent, at least in some embodiments. For example, when the composition is exposed to an acidic condition (e.g., pH of less than 3.5), a binding affinity between the polyphenol and the enzyme is substantially greater than a binding affinity between the polyphenol and the metal ion. In some embodiments, a majority of the polyphenol is associated with the enzyme, such that the enzyme is inhibited from converting a sugar. When the composition is exposed to a relatively high pH (e.g., a neutral pH), the binding affinity between the polyphenol and the enzyme may be substantially less than the binding affinity between the polyphenol and the metal ion. In some instances, the polyphenol may associate (e.g., chelate) with the metal ions and the enzyme is no longer inhibited from converting the sugar.

The composition described above may be beneficial, for example, for use within an acidic food product (e.g., a juice) containing a sugar. For example, without wishing to be bound by any theory, prior to consumption and/or the instant upon consumption, the polyphenol may preferentially associate with the enzymes (as opposed to the metal ions) in the acidic food product such that the sugar within the food product remains unconverted. As such, the authentic taste (e.g., sweetness) of the food product may be maintained upon consumption. During and/or after consumption, as the composition enters into the digestive tract (e.g., intestine), the composition may be destabilized by the digestive juice (e.g., intestinal juice) due to a pH condition (e.g., relatively neutral pH in the intestine). As a result, the polyphenol may preferentially bind to the metal ions (as opposed to the enzymes) and thus freeing the enzyme to convert the sugar in the food product in the digestive tract. Alternatively or additionally, the metal ion may directly bind with the enzyme in a way that allows the enzyme to bind to and convert sugar in the food product in the digestive tract.

As mentioned above, a composition may comprise a particle comprising an enzyme and an inhibitor described herein. The particle may be used as a carrier for delivery of an inner content (e.g., enzyme, inhibitor, additives, metal ions, etc.) to a location internal of a subject and/or physically protect the inner content from premature exposure to an external environment. The particle, for example, may protect the inner content from gaining contact with a sugar in a food product during storage. As described in more detail below, the particle may comprise a stimuli-responsive material capable of releasing its inner content when subjected to a particular pH and/or a temperature condition (e.g., such as physiological pH and/or temperature) within the GI tract of a subject. The particle may have any of a variety of morphologies and/or types, including, but not limited to, a fluidic droplet, a double emulsion, a microcapsule, and a polymeric particle. Depending on the type or morphology of the particle, the enzyme and inhibitors may be associated with the particles via any of a variety of routes. Such routes include encapsulation, physical or chemical attachment (e.g., conjugation), and/or physical entrapment.

For example, in embodiments in which the particle is a polymeric particle, the enzyme, along with the associated inhibitor, may be attached to and/or entrapped within the particle via any appropriate chemical or physical means. For example, in one set of embodiments, the enzyme may be attached (e.g., via physical adsorption) to a particle using a linker species. In some embodiments, the linker species may be a polymer comprising carboxymethyl cellulose. Any appropriate chemistries (e.g., EDC/NHS chemistry) may be used to bound the enzyme to the linker species.

In one set of embodiments, the enzyme may be attached to a substrate (e.g., a nanocellulose substrate) via an optional linker species and further encapsulated and/or entrapped within a particle. Advantageously, the attachment of the enzyme to the substrate may prevent or limit diffusion of the enzyme into an environment external of the particle, such that the enzyme may be better retained within the particle. The substrate, for example, may have a size larger than the pores size or mesh size of the particle, e.g., such that the substrate-enzyme conjugate exhibit limited (if any) diffusion into an environment external of the particle. In some embodiments, the substrate may comprise a material capable of associating with the mucus layer of the gastrointestinal tract. Alternatively or additionally, the substrate may comprise a fiber that is insoluble at a relatively high pH (e.g., pH of greater than 6, greater than 6.6, etc.). In some cases, the substrate comprises cellulose (e.g., nanocellulose and/or micro-cellulose). In some cases, the substrate comprises nanocellulose. Examples of nanocellulose include, but are not limited to, nanofiber cellulose and nanocrystal cellulose. While FIG. 1 illustrates an embodiment in which the enzyme is not attached to a substrate, it should be understood that not all embodiments described are so limiting, and in other embodiments, the enzyme may be attached to a substrate (e.g., nanocellulose) and encapsulated within the particle.

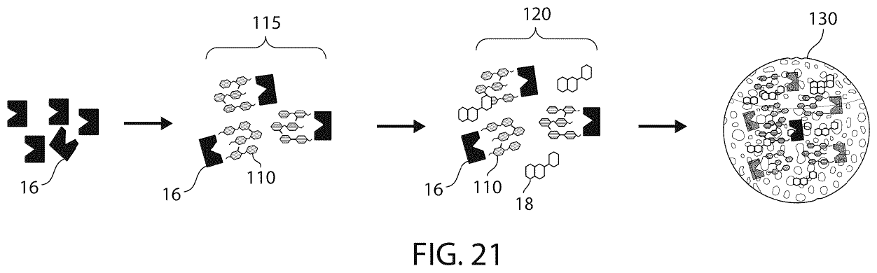

For example, a schematic of a non-limiting example of a substrate-enzyme conjugate is illustrated in FIG. 21. As shown, an enzyme 16 may be conjugated with a substrate 110, thereby forming a substrate-enzyme conjugate 115. In some embodiments, compared to non-conjugated enzymes, substrate-enzyme conjugates may offer several advantages, e.g., such as resulting in a more stable and effective composition. For instance, the presence of the substrate may lead to better retention of enzymes within particles and protect the enzyme from harsh fluid environment within the gastrointestinal tract (e.g., intestine). In some cases, the substrate may advantageously lead to prolonged retention of enzymes in a location (e.g., gastrointestinal tract) within a subject, thereby allowing for a higher conversion (e.g., polymerization) of sugar at the location within the subject compared to non-conjugated enzymes.

In some embodiments, the substrate-enzyme complex may be further combined with an inhibitor (e.g., a polyphenol). For example, as shown in FIG. 21, the substrate-enzyme conjugate 115 may be combined with an inhibitor 18 to form a substrate-enzyme-inhibitor complex 120. In some embodiments, the presence of the inhibitor may provide inhibition and control of enzymatic activity. For example, the inhibitor may be capable of inhibiting and/or activating the enzyme in response to a change in a condition (e.g., pH and/or ionic strength). In some embodiments, a particle may be optionally employed to encapsulate the substrate-enzyme-inhibitor complex. For example, as shown in FIG. 21, the substrate-enzyme-inhibitor complex 120 may be further contained within a particle 130. As described elsewhere herein, the particle may allow for controlled release of the enzyme and associated complex upon a change in an environmental condition (e.g., ionic strength, pH and/or temperature).

In some embodiments, the particle may be used to encapsulate an enzyme (optionally attached to a substrate (e.g., nanocellulose)) and an inhibitor described herein. In some embodiments, the particle may be a microcapsule, a double emulsion, or a fluidic droplet comprising one or more shells that comprise a polymer, a lipid (e.g., a phospholipid), and/or a protein. In some embodiments, the particle comprises a fluidic droplet having one or more shells comprising a phospholipid and/or a protein. In one set of embodiments, the particle comprises a fluidic droplet comprising an inner shell comprising a polymer and another shell comprising a protein and/or a phospholipid.

Any suitable particle may be used. For instance, the particle may be one that is substantially porous, or one that contains a degradable outer shell, e.g., encapsulating the enzyme, inhibitor, etc., such as are described herein. In some embodiments, the particle may be a stimuli-responsive particle. For instance, upon reaching a predetermined location (e.g., GI tract) internal of a subject, the particle may respond to a change in pH and/or temperature at the location, such particle at least partially degrades and/or swells. In some cases, the inner content (e.g., enzyme, inhibitors, etc.) of the particle may be released and/or gain direct contact with a sugar at the location such that the sugar can be converted by the enzyme. Non-limiting examples of such particles can be seen, for example, in Int. Pat. Apl. Pub. No. WO 2013/006661, incorporated herein by reference.

The particle may comprise any of a variety of appropriate polymers. Non-limiting examples of such polymers include, but are not limited to, prebiotic polymers, cellulose (e.g., nanocellulose (e.g., nanofiber cellulose)), amylose, gelatin, cellulose derivatives, polyvinyl alcohol, pectin, alginate, agarose, glucomannan, phytagel, pullulan, inulin, xanthan gum. Non-limiting examples of pH-responsive polymers include pectin, alginate, carrageenan, xanthan gum, guar gum, gum Arabic, carboxy methylcellulose. Non-limiting examples of temperature-responsive polymers include gelatin, collagen, methylcellulose, kappa-carrageenan.

In one set of embodiments, the particle is formed from a fluidic droplet containing an enzyme and an inhibitor. In some embodiments, the particle may be formed from the fluidic droplet, e.g., containing the enzyme and the inhibitor distributed within the particle. In addition, in some embodiments, the particle comprises a shell surrounding a fluid, where the shell is configured to prevent fluidic communication between the enzyme and a sugar external of the particle.

FIG. 1 illustrates such an embodiment, as a non-limiting example. As shown, particle 10 comprises a fluidic droplet 12 and a shell 14 surrounding fluidic droplet. The fluid droplet 12 may contain an enzyme 16, an inhibitor 18 associated with the enzyme, and an additive 20 contained within the fluid. As shown, the shell 14 surrounding the fluid is configured to prevent fluidic communication between the enzyme 16 and the sugar 11 external of the particle.

In some embodiments, the particle comprises a pH-responsive and/or temperature-responsive shell. The shell may comprise any of a variety of polymers described herein. In one set of embodiments, the shell may comprise agarose, pectin, or mixture of agarose and pectin. In some cases, the shell may comprise agarose cross-linked with pectin. While FIG. 1 shows an embodiment in which the particle comprises a single shell, e.g., surrounding a fluid, it should be understood that not all embodiments described are so limiting, and in other embodiments, the particle may comprise more than one shell, such as two or more shells. For instance, in one set of embodiments, the particle may comprise a first shell comprising agarose, and a second shell comprising pectin.

In some embodiments, the pH-responsive and/or temperature responsive shell may at least partially dissociate and/or swell when exposed to a change in pH and/or temperature at a location (e.g., GI tract) internal to a subject. As the shell dissociates and/or swells, the enzyme within the fluidic droplet may gain access to a sugar external to the particle and the sugar may be converted by the enzyme.

FIGS. 1-3 can be used to illustrate such an embodiment. As shown in FIG. 1, when particle 10 is exposed to a first condition (e.g., a pH and/or temperature in a food product), particle 10 is intact and the inner content is prevented from gaining contact with sugar 11. As particle 10 is introduced to a second condition (e.g., a pH and/or temperature in the GI tract), shell 14 of particle 10 is configured to at least partially dissociate (e.g., as shown in FIG. 2). In accordance with some embodiments, upon exposure to the second condition (e.g., a pH in the GI tract), inhibitor 16 may dissociate from enzyme 16 and associate with additive 20. Accordingly, as the shell of the particle dissociates, the enzyme within the particle may gain access to a sugar (e.g., a monosaccharide or disaccharide) and the sugar may be converted (e.g., polymerized) into a non-digestible form (e.g., polysaccharide). Referring to FIG. 3, as enzyme 16 gains contact with sugar 11, sugar 11 may be converted by enzyme 16.

In some embodiments, the particle (or shell of the particle) may be configured to at least partially degrade and/or swell at a temperature of at least 32.5° C. (e.g., at least 35° C., at least 37° C., at least 40° C., or at least 45° C.). In some embodiments, the particle (or shell of the particle) may be configured to at least partially degrade and/or swell at a temperature of no more than 60° C. (e.g., no more than 55° C., no more than 50° C., no more than 45° C., no more than 40° C., no more than 35° C., no more than 30° C.). Combination of the above-referenced ranges are possible (e.g., at least 30° C. and no more than 60° C., at least 35° C. and no more than 50° C., or at least 37° C. and no more than 40° C.). Other ranges are also possible.

In some embodiments, the particle (or shell of the particle) may be configured to at least partially degrade and/or swell at a pH of at least 5 (e.g., at least 5.5, at least 6, at least 6.5, at least 6.8, or at least 7). In some embodiments, the particle (or shell of the particle) may be configured to at least partially degrade and/or swell at a temperature of no more than a pH of 7.5 (e.g., no more than 7.5, no more than 7, no more than 6.8, no more than 6.5, no more than 6, or no more than 5.5). Combination of the above-referenced ranges are possible (e.g., at least 5 and no more than 7.5). Other ranges are also possible.

While FIGS. 1-3 show an embodiment in which the composition comprises a particle encapsulating and/or entrapping the enzyme and the associated inhibitor, it should be understood that not all embodiments described are so limiting, and in other embodiments, the composition lacks a particle encapsulating and/or entrapping the enzyme and the associated inhibitor. For instance, in one set of embodiments, the composition comprises an enzyme and an inhibitor that are not encapsulated and/or entrapped in a particle. For example, in one set of embodiments, the composition may comprise one or more enzymes conjugated to a substrate (e.g., nanocellulose) and optionally complexed to an inhibitor. The composition may optionally comprise any of the additives (e.g., minerals, metal ions, etc.) described herein. In some such embodiments, the composition may be used in and/or with any of variety of food products containing a sugar, e.g., such as a dry food product (e.g., chocolate, baked goods, etc.) containing a sugar.

Similarly, while FIGS. 1-3 show an embodiment in which the composition comprises a particle comprising an enzyme, an inhibitor, and an additive, it should be understood that not all embodiments described are so limiting. As noted, in some embodiments, a composition may comprise a particle comprising an enzyme and does not include an inhibitor and/or an additive described herein. For example, the composition may include a particle that includes an enzyme, or an enzyme conjugated to a substrate (e.g., an insoluble polymer such as nanocellulose), for example, a particle that is also free of inhibitor. Such a composition may be used in and/or with any of a variety of food products that contain a sugar, as described elsewhere herein.

In some embodiments, the composition further comprises a supplement comprising a probiotic. The supplement may be encapsulated and/or entrapped within the particle(s) within the composition. In some embodiments, the supplement comprises a probiotic capable of metabolizing a prebiotic. As noted above, the prebiotic may be an indigestible sugar (e.g., fructooligosaccharides, inulin, levan) formed via polymerization reaction of a sugar (e.g., monosaccharide and/or disaccharide) catalyzed by an enzyme within the composition, as described above.

In one set of embodiments, the composition comprises a particle comprising an enzyme capable of converting a sugar (within a food product) into a prebiotic, an inhibitor, a supplement comprising a probiotic, and optional additives (e.g., metal ions, etc.) described herein. Such a composition may advantageously allow for a reduction in simple sugars (e.g., monosaccharide and/or disaccharide) within a food product after consumption, and simultaneously promote a subject's gut health when the probiotic (and the prebiotics formed from conversion of simple sugars) has been released in the GI tract.

Certain embodiments are directed to ingesting (e.g., swallowing) a composition described herein. In some embodiments, the composition may be combined with a food product containing a sugar prior to being consumed (e.g., swallowed) by a subject. Once consumed and delivered to a predetermined location (e.g., GI tract) internal of a subject, a pH and/or temperature at the location may trigger the composition to convert the sugar from a digestible form (e.g., monosaccharide or disaccharide) into a non-digestible or less digestible form (e.g., a polysaccharide). In some embodiments, the location internally of the subject is the colon, the duodenum, the ileum, the jejunum, or the stomach. In addition, in some embodiments, the composition need not be combined with a food product. For example, the composition may be ingested separately by a subject, e.g., before or eating a food product.

Some embodiments are generally directed to a composition comprising a digestive juice comprising a particle such as is described herein. For example, in accordance with certain embodiments, the particle comprises an enzyme capable of polymerizing a sugar to produce a polymer, and an inhibitor capable of inhibiting the enzyme from polymerizing the sugar. The particle may further comprise an additive described herein. In some embodiments, the digestive juice comprises gastric juice or the intestinal juice. In some cases, the composition may be present in an in vitro setting (e.g., for laboratory testing purposes). In addition, such a composition may be created within a subject, e.g., upon ingestion of particles such as those described herein.

In the presence of digestive juice, the particle may at least partially dissociate and/or swell, such that the enzyme within the particle may convert a sugar external to the particle. In some embodiments, the digestive juice may have a pH and/or temperature that assists with the dissociation of the particle and the enzymatic conversion of the sugar. For example, in one set of embodiments, the digestive juice is the intestinal juice. Intestinal juice typically has a neutral pH and is associated with a temperature of about or equal to 37° C. (temperature in the intestine). In the presence of the intestinal juice, an associated inhibitor-enzyme complex (e.g., as shown in FIG. 1) may dissociate and an inhibitor-additive complex may form (e.g., as shown in FIG. 2). The particle may simultaneously at least partially dissociate and/or swell, thus allowing the sugar to be converted by the enzyme (e.g., as shown in FIGS. 2-3).

In some embodiments, the digestive juice may further comprise a particular amount of bile salt that can assist with the enzymatic conversion of a sugar. The bile salt may associate with the inhibitor via a combination of hydrophobic effect and/or hydrogen bonding, thus rendering the inhibitor incapable of binding to the enzyme. As such, the inhibitor may dissociate from the enzyme and allow the enzyme to convert the sugar. Without wishing to be bound by theory, it is believed that the bile salt may synergistically interact with the additive described herein (e.g., a metal ion) to facilitate enzymatic conversion of a sugar.

In some embodiments, a composition described herein may be triggered to convert a sugar by any of a variety of pH conditions. For instance, the composition may be triggered to convert a sugar at any of a pH of at least 3.5, at least 4, at least 4.5, at least 5, at least 5.5, at least 6, at least 6.5, at least 7, at least 7.2, at least 7.4, at least 7.6, at least 7.8, or at least 8. In some instances, the composition described herein may be triggered to convert a sugar at any of a pH of no more than 9, no more than 8.5, no more than 8, no more than 7.8, no more than 7.6, no more than 7.4, no more than 7.2, no more than 7, no more than 6.5, no more than 6, no more than 5.5, no more than 5, no more than 4.5, or no more than 4. Combination of the above-referenced ranges are possible (e.g., at least 3.5 and no more than 9, at least 3.5 and no more than 8, or at least 6 and no more than 7.4). Other ranges are also possible.

In some embodiments, a composition described herein may be triggered to convert a sugar by any of a variety of temperatures. For instance, the composition may be triggered to convert a sugar at any of a temperature of at least 30° C., at least 35° C., at least 37° C., at least 40° C., at least 45° C., at least 50° C., at least 55° C. In some instances, the composition may be triggered to convert a sugar at any of a temperature of no more than 60° C., no more than 55° C., no more than 50° C., no more than 45° C., no more than 40° C., no more than 37° C., or no more than 35° C. Combination of the above-referenced ranges are possible (e.g., at least 30° C. and no more than 60° C., at least 35° C. and no more than 50° C., or at least 37° C. and no more than 50° C.). Other ranges are also possible. In some cases, the temperature may be selected such that the trigger occurs after ingestion (e.g., by a subject). In some embodiments, however, higher temperatures may be used, e.g., upon exposure to a hot beverage, such as coffee or tea. In some embodiments, a composition described herein may be triggered to convert a sugar by a combination of pH and temperatures in one or more of the ranges described above.

The composition may be combined with any of a variety of food products containing a sugar. Non-limiting examples of food products include fruit or vegetable juice, fruit or vegetable puree, condiments, solid canned food, food seasonings, confectionary, crystalline drink powder, powder sauces, baking goods, creams, salads and fruit dressings, dairy products.

In some embodiments, a method of making a composition is disclosed herein. In some embodiments, an enzyme is combined (e.g., associated) to an inhibitor to form a complex, after which the complex is associated (e.g., attached, encapsulated, or entrapped) with a particle within the composition.

The particle may have any of a variety of particle sizes or shapes. In some embodiments, the particles have an average diameter of at least 50 nm, at least 100 nm, at least 200 nm, at least 500 nm, at least 1 micrometers, at least 5 micrometers, at least 10 micrometers, at least 25 micrometers, at least 50 micrometers, at least 75 micrometers, at least 100 micrometers, at least 250 micrometers, or at least 500 micrometers. In some embodiments, the particles may have an average size of no more than 750 micrometers, no more than 500 micrometers, no more than 250 micrometers, no more than 100 micrometers, no more than 75 microns, no more than 50 micrometers, no more than 25 micrometers, no more than 10 micrometers, no more than 5 micrometers, no more than 1 micrometers, no more than 500 nm, no more than 250 nm, no more than 100 nm, or no more than 50 nm. Combination of the above-referenced ranges are possible (e.g., at least 50 nm and no more than 750 micrometers, or at least 10 micrometer and no more than 100 micrometers). Other ranges are also possible. For example, in embodiments in which the particle comprises a supplement comprising a probiotic, the particle may have a particle size of up to 1 mm or higher.

The particles described herein may be fabricated via any of a variety of appropriate methods. Non-limiting examples of such methods include microfluidics, sonication, spray drying, emulsification, acoustophoretic printing, piezoelectric droplet generators, extrusion, co-extrusion, etc. In particular, one non-limiting example of a method of using microfluidics to form a particle comprising a fluidic droplet and a shell encapsulating the fluidic droplet (as shown in FIG. 1) is described in more detail below.

In some embodiments, the particle comprising a fluid droplet and a shell (e.g., as shown in FIG. 1) may be formed as a part of an emulsion. For example, the emulsion may be a double emulsion (e.g., W₁/W₂/O) comprising a first aqueous phase (W₁) forming the fluidic droplet, a second aqueous phase (W₂) forming the shell around the fluidic droplet, and an immiscible continuous phase (e.g., an oil (O)) as the carrying fluid. In some embodiments, the first aqueous phase comprises an enzyme or enzyme conjugated to a substrate (e.g., carboxymethylcellulose, nanocellulose), an inhibitor, and optionally additives described herein. In some embodiments, the second aqueous phase comprises a polymer precursor (e.g., agarose and/or pectin) that is capable of being crosslinked to form a polymeric shell. The first aqueous phase may be substantially immiscible with the second aqueous phase in part due to a significant difference in their viscosities.

In one aspect of the present disclosure, emulsions are formed by flowing two, three, or more fluids through a system of conduits. The system may be a microfluidic system. “Microfluidic,” as used herein, refers to a device, apparatus or system including at least one fluid channel having a cross-sectional dimension of less than about 1 millimeter (mm), and in some cases, a ratio of length to largest cross-sectional dimension of at least 3:1. One or more conduits of the system may be a capillary tube. In some cases, multiple conduits are provided, and in some embodiments, at least some are nested, as described herein. The conduits may be in the microfluidic size range and may have, for example, average inner diameters, or portions having an inner diameter, of less than about 1 millimeter, less than about 300 micrometers, less than about 100 micrometers, less than about 30 micrometers, less than about 10 micrometers, less than about 3 micrometers, or less than about 1 micrometer, thereby providing droplets having comparable average diameters. One or more of the conduits may (but not necessarily), in cross section, have a height that is substantially the same as a width at the same point. Conduits may include an orifice that may be smaller, larger, or the same size as the average diameter of the conduit. For example, conduit orifices may have diameters of less than about 1 mm, less than about 500 micrometers, less than about 300 micrometers, less than about 200 micrometers, less than about 100 micrometers, less than about 50 micrometers, less than about 30 micrometers, less than about 20 micrometers, less than about 10 micrometers, less than about 3 micrometers, etc. In cross-section, the conduits may be rectangular or substantially non-rectangular, such as circular or elliptical. The conduits of the present disclosure can also be disposed in or nested in another conduit, and multiple nestings are possible in some cases. In some embodiments, one conduit can be concentrically retained in another conduit and the two conduits are considered to be concentric. In other embodiments, however, one conduit may be off-center with respect to another, surrounding conduit. By using a concentric or nesting geometry, the inner and outer fluids, which are typically miscible, may avoid contact, which can facilitate great flexibility in making multiple emulsions and in devising techniques for encapsulation and polymerosome formation. For example, this technique allows for fabrication of core-shell structure, and these core-shell structures can be converted into capsules.

A “channel,” as used herein, means a feature on or in an article (substrate) that at least partially directs flow of a fluid. The channel can have any cross-sectional shape (circular, oval, triangular, irregular, square or rectangular, or the like) and can be covered or uncovered. In embodiments where it is completely covered, at least one portion of the channel can have a cross-section that is completely enclosed, or the entire channel may be completely enclosed along its entire length with the exception of its inlet(s) and/or outlet(s). A channel may also have an aspect ratio (length to average cross sectional dimension) of at least 2:1, more typically at least 3:1, 5:1, 10:1, 15:1, 20:1, or more. An open channel generally will include characteristics that facilitate control over fluid transport, e.g., structural characteristics (an elongated indentation) and/or physical or chemical characteristics (hydrophobicity vs. hydrophilicity) or other characteristics that can exert a force (e.g., a containing force) on a fluid. The fluid within the channel may partially or completely fill the channel. In some cases where an open channel is used, the fluid may be held within the channel, for example, using surface tension (i.e., a concave or convex meniscus).

The channel may be of any size, for example, having a largest dimension perpendicular to fluid flow of less than about 5 mm or 2 mm, or less than about 1 mm, or less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm. In some cases the dimensions of the channel may be chosen such that fluid is able to freely flow through the article or substrate. The dimensions of the channel may also be chosen, for example, to allow a certain volumetric or linear flowrate of fluid in the channel. Of course, the number of channels and the shape of the channels can be varied by any method known to those of ordinary skill in the art. In some cases, more than one channel or capillary may be used. For example, two or more channels may be used, where they are positioned inside each other, positioned adjacent to each other, positioned to intersect with each other, etc.

As the systems described herein may be three-dimensional microfluidic devices, e.g., having concentric conduit arrangements, a fluid (of any nesting level of a multiple emulsion) can be completely shielded from a surrounding fluid in certain embodiments. This may reduce or eliminate problems that can occur in other systems, when the fluids may contact each other at or near a solid surface, such as in a two-dimensional system.

In some embodiments, a flow pathway can exist in an inner conduit and a second flow pathway can be formed in a coaxial space between the external wall of the interior conduit and the internal wall of the exterior conduit, as discussed in detail below. The two conduits may be of different cross-sectional shapes in some cases. In one embodiment, a portion or portions of an interior conduit may be in contact with a portion or portions of an exterior conduit, while still maintaining a flow pathway in the coaxial space. Different conduits used within the same device may be made of similar or different materials. For example, all of the conduits within a specific device may be glass capillaries, or all of the conduits within a device may be formed of a polymer, for example, polydimethylsiloxane, as discussed below.

A geometry that provides coaxial flow can also provide hydrodynamic focusing of that flow, according to certain embodiments of the disclosure. Many parameters of the droplets, including any suitable nesting layer in a multiple emulsion droplet, can be controlled using hydrodynamic focusing. For instance, droplet diameter, outer droplet thickness and the total number of inner droplets per droplet can be controlled.

Parameters for controlling emulsion or droplet formation can be controlled by adjusting, for example, the system geometry, and/or the flowrate of any of the fluids used to form the emulsion or droplet.

A variety of materials and methods, according to certain aspects of the disclosure, can be used to form systems, such as microfluidic systems, (such as those described above) able to produce the droplets described herein. In some cases, the various materials selected lend themselves to various methods. For example, various components of the disclosure can be formed from solid materials, in which the channels can be formed via micromachining, film deposition processes such as spin coating and chemical vapor deposition, laser fabrication, photolithographic techniques, etching methods including wet chemical or plasma processes, and the like. See, for example, Scientific American, 248:44-55, 1983 (Angell, et al). In one embodiment, at least a portion of the fluidic system is formed of silicon by etching features in a silicon chip. Technologies for precise and efficient fabrication of various fluidic systems and devices of the disclosure from silicon are known. In another embodiment, various components of the systems and devices of the disclosure can be formed of a polymer, for example, an elastomeric polymer such as polydimethylsiloxane (“PDMS”), polytetrafluoroethylene (“PTFE” or Teflon®), or the like.

Different components can be fabricated of different materials. For example, a base portion including a bottom wall and side walls can be fabricated from an opaque material such as silicon or PDMS, and a top portion can be fabricated from a transparent or at least partially transparent material, such as glass or a transparent polymer, for observation and/or control of the fluidic process. Components can be coated so as to expose a desired chemical functionality to fluids that contact interior channel walls, where the base supporting material does not have a precise, desired functionality. For example, components can be fabricated as illustrated, with interior channel walls coated with another material. Material used to fabricate various components of the systems and devices of the disclosure, e.g., materials used to coat interior walls of fluid channels, may desirably be selected from among those materials that will not adversely affect or be affected by fluid flowing through the fluidic system, e.g., material(s) that is chemically inert in the presence of fluids to be used within the device.

In one embodiment, various components of the disclosure are fabricated from polymeric and/or flexible and/or elastomeric materials, and can be conveniently formed of a hardenable fluid, facilitating fabrication via molding (e.g. replica molding, injection molding, cast molding, etc.). The hardenable fluid can be essentially any fluid that can be induced to solidify, or that spontaneously solidifies, into a solid capable of containing and/or transporting fluids contemplated for use in and with the fluidic network. In one embodiment, the hardenable fluid comprises a polymeric liquid or a liquid polymeric precursor (i.e. a “prepolymer”). Suitable polymeric liquids can include, for example, thermoplastic polymers, thermoset polymers, or mixture of such polymers heated above their melting point. As another example, a suitable polymeric liquid may include a solution of one or more polymers in a suitable solvent, which solution forms a solid polymeric material upon removal of the solvent, for example, by evaporation. Such polymeric materials, which can be solidified from, for example, a melt state or by solvent evaporation, are well known to those of ordinary skill in the art. A variety of polymeric materials, many of which are elastomeric, are suitable, and are also suitable for forming molds or mold masters, for embodiments where one or both of the mold masters is composed of an elastomeric material. A non-limiting list of examples of such polymers includes polymers of the general classes of silicone polymers, epoxy polymers, and acrylate polymers. Epoxy polymers are characterized by the presence of a three-membered cyclic ether group commonly referred to as an epoxy group, 1,2-epoxide, or oxirane. For example, diglycidyl ethers of bisphenol A can be used, in addition to compounds based on aromatic amine, triazine, and cycloaliphatic backbones. Another example includes the well-known Novolac polymers. Non-limiting examples of silicone elastomers suitable for use according to the disclosure include those formed from precursors including the chlorosilanes such as methylchlorosilanes, ethylchlorosilanes, phenylchlorosilanes, etc.

Silicone polymers are preferred in one set of embodiments, for example, the silicone elastomer polydimethylsiloxane. Non-limiting examples of PDMS polymers include those sold under the trademark Sylgard by Dow Chemical Co., Midland, MI, and particularly Sylgard 182, Sylgard 184, and Sylgard 186. Silicone polymers including PDMS have several beneficial properties simplifying fabrication of the microfluidic structures of the disclosure. For instance, such materials are inexpensive, readily available, and can be solidified from a prepolymeric liquid via curing with heat. For example, PDMSs are typically curable by exposure of the prepolymeric liquid to temperatures of about, for example, about 65° C. to about 75° C. for exposure times of, for example, about an hour. Also, silicone polymers, such as PDMS, can be elastomeric, and thus may be useful for forming very small features with relatively high aspect ratios, necessary in certain embodiments of the disclosure. Flexible (e.g., elastomeric) molds or masters can be advantageous in this regard.

One advantage of forming structures such as microfluidic structures of the disclosure from silicone polymers, such as PDMS, is the ability of such polymers to be oxidized, for example by exposure to an oxygen-containing plasma such as an air plasma, so that the oxidized structures contain, at their surface, chemical groups capable of cross-linking to other oxidized silicone polymer surfaces or to the oxidized surfaces of a variety of other polymeric and non-polymeric materials. Thus, components can be fabricated and then oxidized and essentially irreversibly sealed to other silicone polymer surfaces, or to the surfaces of other substrates reactive with the oxidized silicone polymer surfaces, without the need for separate adhesives or other sealing means. In most cases, sealing can be completed simply by contacting an oxidized silicone surface to another surface without the need to apply auxiliary pressure to form the seal. That is, the pre-oxidized silicone surface acts as a contact adhesive against suitable mating surfaces. Specifically, in addition to being irreversibly sealable to itself, oxidized silicone such as oxidized PDMS can also be sealed irreversibly to a range of oxidized materials other than itself including, for example, glass, silicon, silicon oxide, quartz, silicon nitride, polyethylene, polystyrene, glassy carbon, and epoxy polymers, which have been oxidized in a similar fashion to the PDMS surface (for example, via exposure to an oxygen-containing plasma). Oxidation and sealing methods useful in the context of the present disclosure, as well as overall molding techniques, are described in the art, for example, in an article entitled “Rapid Prototyping of Microfluidic Systems and Polydimethylsiloxane,” Anal. Chem., 70:474-480, 1998 (Duffy, et al.), incorporated herein by reference.

In some embodiments, certain microfluidic structures of the disclosure (or interior, fluid-contacting surfaces) may be formed from certain oxidized silicone polymers. Such surfaces may be more hydrophilic than the surface of an elastomeric polymer. Such hydrophilic channel surfaces can thus be more easily filled and wetted with aqueous solutions.

In one embodiment, a bottom wall of a microfluidic device of the disclosure is formed of a material different from one or more side walls or a top wall, or other components. For example, the interior surface of a bottom wall can comprise the surface of a silicon wafer or microchip, or other substrate. Other components can, as described above, be sealed to such alternative substrates. Where it is desired to seal a component comprising a silicone polymer (e.g. PDMS) to a substrate (bottom wall) of different material, the substrate may be selected from the group of materials to which oxidized silicone polymer is able to irreversibly seal (e.g., glass, silicon, silicon oxide, quartz, silicon nitride, polyethylene, polystyrene, epoxy polymers, and glassy carbon surfaces which have been oxidized). Alternatively, other sealing techniques can be used, as would be apparent to those of ordinary skill in the art, including, but not limited to, the use of separate adhesives, thermal bonding, solvent bonding, ultrasonic welding, etc.

Fructosyltransferases that may be useful include, but are not limited to, those classified as EC.2.4.1.99. Such enzymes may exhibit transferase activity. Such enzymes are sometimes also called beta-fructofuranosidases. Beta-fructofuranosidases also include hydrolytic enzymes classified as EC.3.2.1.26. Fructosyltransferases include any enzyme capable of catalyzing the transfer reaction and the use of this term in no way restricts the scope of the disclosure.

Fructosyltransferases used in the disclosure may, in some embodiments, be derived from plant sources such as asparagus, sugar beet, onions, Jerusalem artichokes, and chicory root.

Fructosyltransferase may also be derived from fungal sources, such as Aspergillus, Aureobasidium and Fusarium. More specific examples include Aspergillus japonicus, such as CCRC 38011; Aspergillus niger, such as ATCC 20611; Aspergillus foetidus (such as NRRL 337); Aspergillus aculeatus; Aureobasidium pullulans, such as ATCC 9348, ATCC 12535; and ATCC 15223.

Fructosyltransferases additionally may be derived from bacterial sources, such as Arthrobacter.

In some instances, the fructosyltransferase may be a variant of a naturally occurring fructosyltransferase. Reference is made to U.S. Pat. No. 6,566,111, wherein a beta-fructo-furanosidase was genetically engineered to improve the productivity of the enzyme (see also US Patent Application Publication No. 2002/0192771).

A hexosyltransferase (EC 2.4.1) refers to a type of glycosyltransferase that catalyze the transfer of a hexose.

An amylosucrase or sucrose-glucan glucosyltransferase (EC 2.4.1.4) refers to an enzyme that catalyzes the chemical reaction sucrose and (1,4-alpha-D-glucosyl)ₙ to produce D-fructose and (1,4-alphas-D-glucosyl)ₙ₊₁.

A glucansucrase (EC 2.4.5.1) refers to an enzyme in the glycoside hydrolase family that catalyzes the reaction of sucrose into glucose and fructose, and subsequently catalyzes the formation of homopolysaccharides (alpha-glucan polymers).

A “levansucrase” (EC 2.4.1.10) refers to an enzyme that catalyzes a fructosyl transfer from sucrose to a various acceptor molecules producing mainly levan with D-fructofuranosyl residues linked predominantly by beta-2,6 linkage as the main chain with some beta-2,1 branching points. Examples of levansucrase include LevG derived from Lactobacillus gasseri.

A inulosucrase (EC 2.4.1.9) is an enzyme that catalyzes the chemical reaction sucrose and (2,1-beta-D-fructosyl)ₙ to produce glucose and (2,1-beta-D-fructosyl)ₙ₊₁. Examples of inulosucrase include InuGB derived from Lactobacillus gasseri.

A sucrose:fructose fructosyltransferase is a glycosyltransferase enzyme. Sucrose:fructose fructosyltransferase may be derived from Echinops bannaticus (Globe thistle) or Cichorium intybus (Chicory root).

A fructose:fructose fructosyltransferase is a glycosyltransferase enzyme. Fructose:fructose fructosyltransferase may be derived from Cichorium intybus (Chicory root).

A glucose oxidase (EC 1.1.3.4) is an oxidoreductase that catalyzes the oxidation of glucose to hydrogen peroxide and D-glucono-δ-lactone. In the presence of H₂O, glucose oD-glucono-δ-lactone may be spontaneously converted to gluconic acid.

A fructose dehydrogenase (EC 1.1.99.11) is an enzyme that catalyzes the chemical reaction of D-fructose to dehydro-D-fructose.

A glucose-fructose oxidoreductase (EC 1.1.99.28) is an enzyme that catalyzes the chemical reaction of D-glucose and D-fructose to D-gluconolactone and D-glucitol.

A beta-galactosidase (lactase) is a family of glycoside hydrolase enzymes that catalyzes the hydrolysis of β-galactosides into monosaccharides through the breaking of a glycosidic bond.

An amylase is an enzyme that catalyzes the hydrolysis of starch (Latin amylum) into sugars.

A cellulase is an enzyme produced chiefly by fungi, bacteria, and protozoans that catalyze decomposition of cellulose and of some related polysaccharides.

A laccase is an enzyme that catalyzes the reaction of sucrose and (2,6-beta-D-fructosyl)ₙ, into glucose and (2,6-beta-D-fructosyl)ₙ₊₁. The term “subject,” as used herein, refers to an individual organism such as a human or an animal. In some embodiments, the subject is a mammal (e.g., a human, a non-human primate, or a non-human mammal), a vertebrate, a laboratory animal, a domesticated animal, an agricultural animal, or a companion animal. Non-limiting examples of subjects include a human, a non-human primate, a cow, a horse, a pig, a sheep, a goat, a dog, a cat or a rodent such as a mouse, a rat, a hamster, a bird, a fish, or a guinea pig. Generally, the disclosure is directed toward use with humans. In some embodiments, a subject may demonstrate health benefits, e.g., upon administration of the article and/or the actuating component.

The following applications are each incorporated herein by reference: U.S. patent application Ser. No. 08/131,841, filed Oct. 4, 1993, entitled “Formation of Microstamped Patterns on Surfaces and Derivative Articles,” by Kumar, et al., now U.S. Pat. No. 5,512,131, issued Apr. 30, 1996; U.S. patent application Ser. No. 09/004,583, filed Jan. 8, 1998, entitled “Method of Forming Articles including Waveguides via Capillary Micromolding and Microtransfer Molding,” by Kim, et al., now U.S. Pat. No. 6,355,198, issued Mar. 12, 2002; International Patent Application No. PCT/US96/03073, filed Mar. 1, 1996, entitled “Microcontact Printing on Surfaces and Derivative Articles,” by Whitesides, et al., published as WO 96/29629 on Jun. 26, 1996; International Patent Application No.: PCT/US01/16973, filed May 25, 2001, entitled “Microfluidic Systems including Three-Dimensionally Arrayed Channel Networks,” by Anderson, et al., published as WO 01/89787 on Nov. 29, 2001; U.S. patent application Ser. No. 11/246,911, filed Oct. 7, 2005, entitled “Formation and Control of Fluidic Species,” by Link, et al., published as U.S. Patent Application Publication No. 2006/0163385 on Jul. 27, 2006; U.S. patent application Ser. No. 11/024,228, filed Dec. 28, 2004, entitled “Method and Apparatus for Fluid Dispersion,” by Stone, et al., published as U.S. Patent Application Publication No. 2005/0172476 on Aug. 11, 2005; International Patent Application No. PCT/US2006/007772, filed Mar. 3, 2006, entitled “Method and Apparatus for Forming Multiple Emulsions,” by Weitz, et al., published as WO 2006/096571 on Sep. 14, 2006; U.S. patent application Ser. No. 11/360,845, filed Feb. 23, 2006, entitled “Electronic Control of Fluidic Species,” by Link, et al., published as U.S. Patent Application Publication No. 2007/000342 on Jan. 4, 2007; and U.S. patent application Ser. No. 11/368,263, filed Mar. 3, 2006, entitled “Systems and Methods of Forming Particles,” by Garstecki, et al. In addition, U.S. Provisional Patent Application Ser. No. 63/208,473, filed Jun. 8, 2021, entitled “Systems and Methods for Sugar-Reduction for Food and Other Applications,” is also incorporated herein by reference in its entirety.

The following examples are intended to illustrate certain embodiments of the present disclosure, but do not exemplify the full scope of the disclosure.

### Example 1

This Example describes a composition capable of converting glucose in response to a change in pH and temperature, in accordance with certain embodiments.

The composition contained a plurality of microgel particles. Each of the particles contained glucose oxidase, which is an enzyme capable of catalyzing oxidation of glucose to hydrogen peroxide and D-glucono-delta-lactone, polyphenol associated with glucose oxidase, and minerals comprising metal ions.

The composition was designed to inhibit glucose conversion at a low pH, such that it can be used with an acidic food product (e.g., juice), and convert glucose once the composition reaches the intestine, such that the glucose oxidase in the composition can be activated to convert glucose. The composition was designed such that it can be triggered to convert glucose at a neutral pH and a particular temperature in the intestine.

pH-Triggered Glucose Conversion

Enzymatic activity of glucose oxidase was tested in the presence of polyphenol and metal ions from minerals at different pH. Polyphenol is known to be able to bind to enzymes and also form metal coordination with the metal ions.

The enzyme (e.g, glucose oxidase) was combined with different polyphenols (e.g., tannic acid, EGCG) and metal ions from different minerals (e.g., CaCl₂, FeCl₃, MgCl₂, ZnCl₂) in buffer solutions having two pH conditions. A buffer having a first pH condition of 3.5 was used to simulate juice buffer. A buffer having a second pH condition of 7 was used to simulate intestinal buffer. The effect of bile salt (an intrinsic compound found in the intestine) on enzymatic activity of glucose oxidase was also tested by incorporating it into the simulated pH 7 intestinal buffer. A full experimental design of different compositions is shown in FIG. 4. It should be noted that the enzyme in FIG. 4 is not limited to glucose oxidase, as described in this example. Any appropriate enzymes described elsewherein herein (e.g., inulosucrase, etc.) may be employed using the experimental design in FIG. 4 for sugar reduction.

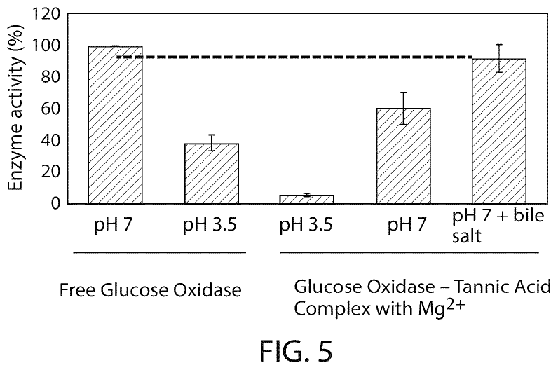

The enzyme activity of glucose oxidase was measured by measuring the capability of glucose oxidase to convert glucose in different experimental conditions. FIG. 5 shows the enzymatic activity of glucose oxidase in the presence of tannic acid and Mg⁺² (from MgCl₂) at different pH conditions. As shown, free glucose oxidase activity was partially inhibited at pH of 3.5. In the presence of tannic acid and Mg⁺², enzymatic activity of glucose oxidase was further inhibited at pH of 3.5. This suggested that at pH of 3.5, tannic acid associated with glucose oxidase and inhibited glucose oxidase from converting glucose. It was observed that tannic acid significantly inhibited glucose oxidase activity at a concentration of 1 micromolar. At a neutral pH of 7, however, enzymatic activity of glucose oxidase was partially recovered. It was hypothesized that at a pH of 7, polyphenol formed metal coordination complexes with metal ions and thus dissociated from the glucose oxidase. At a neutral pH of 7 and in the presence of 0.2 wt % of bile salt, enzymatic activity of glucose oxidase was furthered recovered.

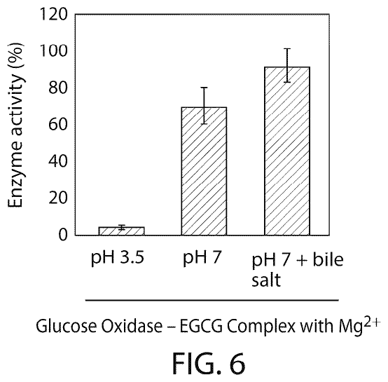

A similar trend in enzymatic acid was observed when a different polyphenol, EGCE, was used instead of tannic acid. FIG. 6 shows the enzymatic activity of glucose oxidase in the presence of EGCE (Epigallocatechin gallate) and Mg⁺² (from MgCl₂) in different pH buffer solutions (e.g., pH 3.5, pH 7, and pH7 with bile salt). As shown, at pH of 3.5, EGCE associated with glucose oxidase and prohibited its enzymatic activity. At pH 7, as EGCE chelates with metal ions and dissociate from glucose oxidase, the enzymatic activity of glucose oxidase was partially recovered. At pH 7 and in the presence of bile salt, further enzymatic activity was recovered.

pH and/or Temperature-Triggered Particle

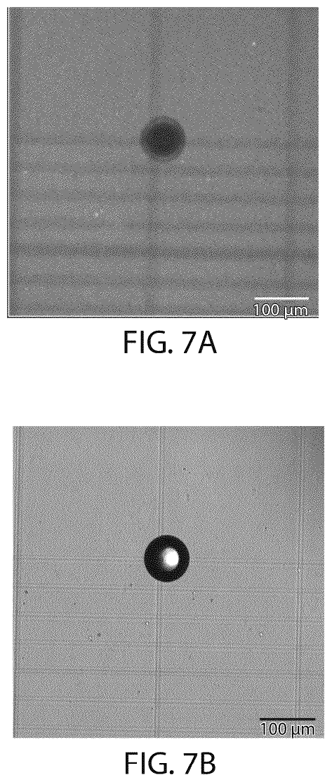

A core-shell structured microgel particle was fabricated using microfluidics (as shown in FIG. 7A). The microgel could be used to encapsulate the glucose oxidase, polyphenol, and minerals comprising metal ions described above. The microgel includes a material that is stable at a low pH, such that the microgel can be used with an acidic food product (e.g., juice), and that can partially degrade or swell at a temperature and/or pH in the GI tract (e.g., stomach, intestine) to allow enzymatic conversion of the sugar in the food product.

A microgel containing a shell of agarose cross-linked with pectin surrounding a fluidic droplet was formed (FIGS. 7A-7B) using microfluidics. A double emulsion (e.g., W₁/W₂/O) included a 2 wt % carboxymethyl cellulose solution (W₁) forming the fluidic droplet, a polymer precursor (W₂) forming the shell, and an immiscible corn oil phase (e.g., an oil (O)) as the carrying fluid. The polymer precursor contained agarose or combination of agarose and pectin. The polymer precursor was then crosslinked to from the polymeric shell.

The stability of polymeric shell could be temperature and/or pH dependent. For example, a microgel having an agarose shell could be triggered to degrade or swell at a temperature (e.g., about 37° C.) in the stomach. For another example, a microgel having an agarose crosslinked with pectin shell exhibited enhanced stability in acidic conditions (e.g., in a juice), and could be triggered to partially dissolve or swell in the stomach at a particular temperature (e.g., about 37° C.) to initiate enzymatic conversion of surrounding sugar. Downstream in the intestine, further degradation of the agarose-crosslinked-with-pectin shell in the higher pH environment would continue to increase enzyme and sugar interactions. Although this example described one specific type of enzymes for sugar reduction and one type of particles for encapsulating the enzymes and associated ingredients, other types of enzymes and particles may be used. Various other enzymes and particles are described in the following examples.

### Example 2

This Example describes a composition comprising fructose dehydrogenase entrapped and/or encapsulated into alginate based particles, in accordance with some embodiments.

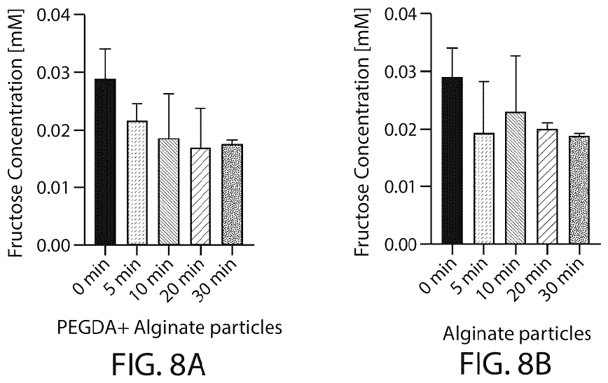

Entrapment and encapsulation of fructose dehydrogenase, e.g., an enzyme for fructose degradation, in alginate based microparticles was demonstrated herein. A 3D printed in-house sprayer was used to spray an alginate based solution containing the enzyme to form sprayed microparticles. The alginate solution contained either sodium alginate or a combination of sodium alginate and polyethylene diacrylate (PEGDA). The sprayed particles were then crosslinked using UV light in the presence of a CaCl₂) solution. Two types of particles containing fructose dehydrogenase were formed: alginate microparticles (FIG. 8B) and alginate-PEGDA particles (FIG. 8A).

The enzymatic activity of entrapped and/or encapsulated fructose dehydrogenase within the two types of particles was measured as a function of free fructose concentration (FIGS. 8A-8B). When the particle (either alginate particle or alginate-PEGDA particle) containing the enzyme was exposed to free fructose, a decrease in free fructose was observed, suggesting that the enzyme within the alginate particle was capable of binding to and degrading fructose.

The particles described in this example may be used to entrap/encapsulate additional ingredients, such as those described in Example 1, including polyphenol, minerals, etc.

### Example 3

This Example describes a composition comprising fructosyltransferase enzymes attached to nanocellulose, in accordance with some embodiments.

Binding of fructosyltransferase enzymes to nanocelloluse particles was demonstrated herein. Fructosyltransferase enzymes are capable of converting sucrose to fructooligosaccharides (FOS), which is a prebiotic that is not digested by human. Two types of fructosyltransferase were studied: levansucrase and inulosucrase. While levansucrase facilitates conversion of sucrose to levan, inulosucrase facilitates conversion of sucrose to inulin.

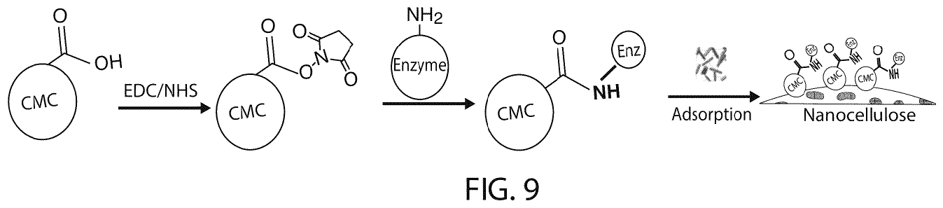

Fructosyltransferase enzymes were attached to nanocellulose fibers using carboxymethyl cellulose (CMC), a molecule that could bind to nanocellulose via adsorption. As shown in FIG. 9, the enzymes could be conjugated to CMC via EDC/NHS chemistry, and the resulting enzyme-CMC complex could be adsorbed onto nanocellulose particles. Alternatively, CMC may be absorbed to the nanocellulose fibers and subsequently conjugated to the enzyme to form nanocellulose-enzyme conjugates. Nanocellulose with attached inulosucrase or levansucrase was successfully manufactured.

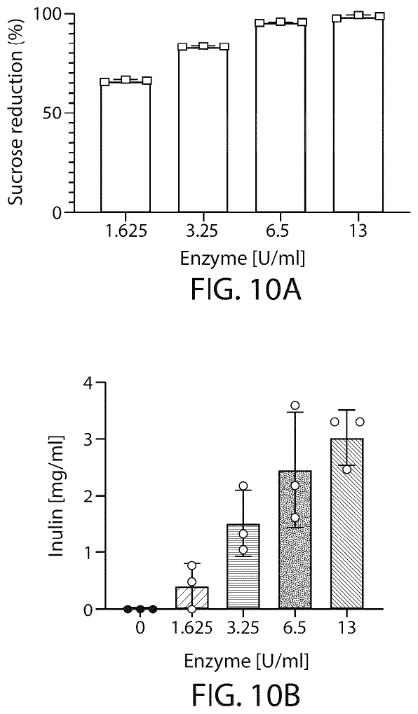

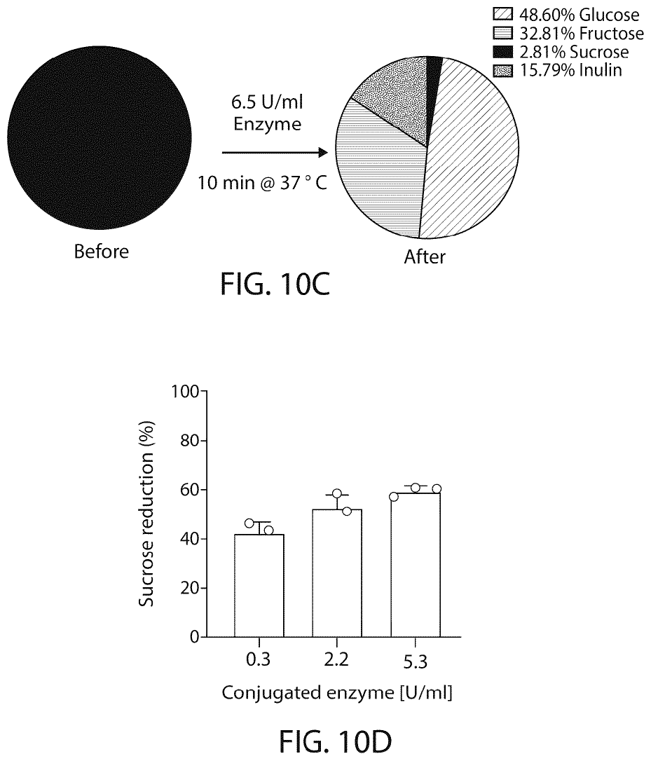

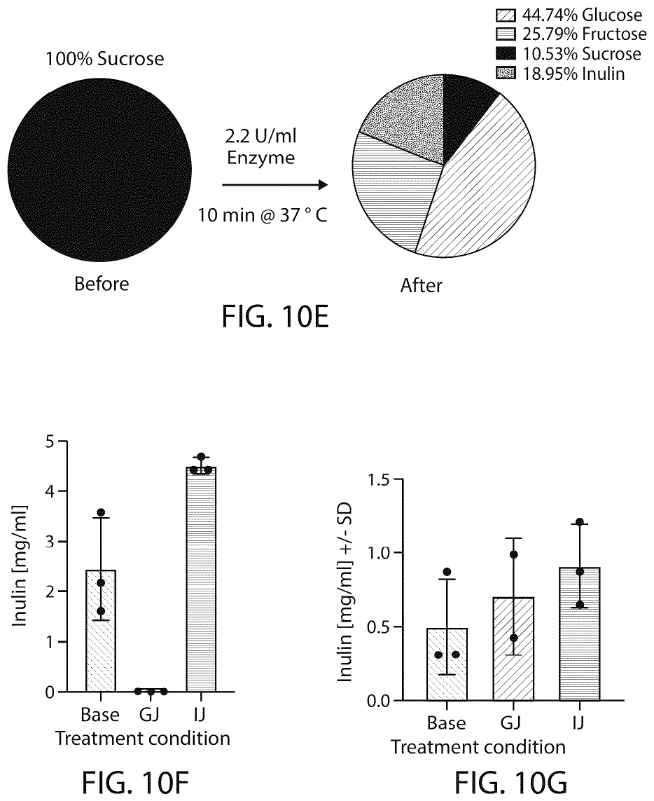

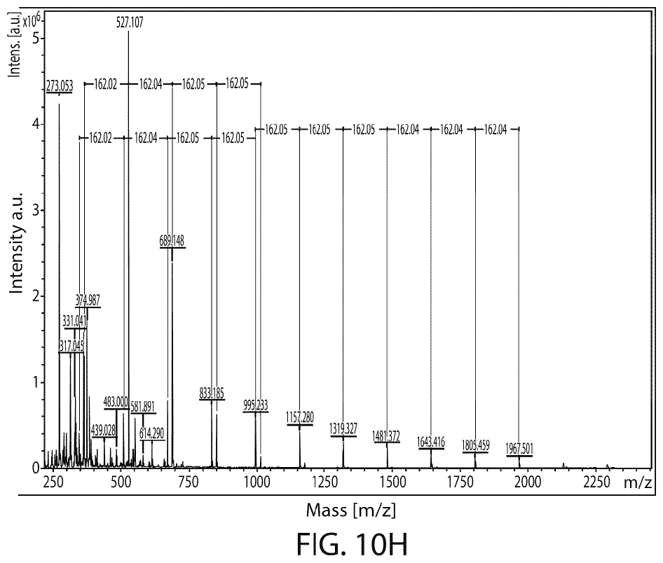

Concentration dependent enzymatic activity of the free inulosucrose enzymes on sugar reduction and inulin formation was evaluated (FIGS. 10A-10B). As shown, the attached inulosucrase led to a significant reduction in the amount of sucrose and increase in inulin formation when present in a concentration of between 1.625 U/mL to 13 U/mL in the composition. A pie chart illustrating sugar and inulin composition of a 0.1 mM sucrose sample before and after treating the sucrose solution with 6.5 U/ml of inulosucrase is presented in FIG. 10C. Concentration dependent enzymatic activity of the conjugated inulosucrose enzymes (i.e., nanocellulose-enzyme conjugates) on sugar reduction was evaluated (FIG. 10D). A pie chart showing sugar and inulin composition of a 0.1 mM sucrose sample before and after treating the sucrose solution with 2.2 U/ml of nanocellulose-enzyme conjugate is presented in FIG. 10E.

The effect of free inulosucrase (not attached to nanocellulose) on sugar conversion in different digestive buffers (having different pH conditions) was first evaluated (FIG. 10F). The enzyme concentration used was 6.5 U/ml and sugar content was evaluated at 37° C. after 10 minutes. Two digestive buffers simulating gastric juice (GJ) at pH 1 and intestinal juice (IJ) at pH 6.2 were used. As shown, more sugar conversion occurred in intestinal juice (IJ).

The effect of inulosucrase (attached onto nanocellulose) on sugar conversion in different digestive buffers (having different pH conditions) was evaluated (FIG. 10G). The enzyme concentration used was 2.2 U/ml and sugar content was evaluated at 37° C. after 10 minutes. Various digestive buffers simulating gastric juice (GJ) at pH 1 and intestinal juice (IJ) at pH 6.2 were used. The formation of inulin was confirmed using MALDI-TOF (FIG. 10H). As shown, different peaks in the MALDI-TOF spectra shows inulin having different numbers of fructose molecules.

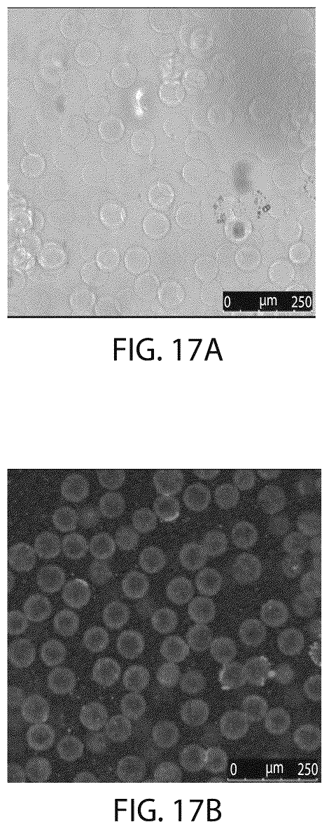

The inulosucrase-nanocellulose complex may be further encapsulated in a particle. For example, the complex may be contained within a fluid droplet that is encapsulated by one or more shells. For example, as shown in FIGS. 17A-17B, the inulosucrase-nanocellulose construct may be contained within a fluidic droplet comprising carboxymethyl cellulose that was encapsulated by a pectin shell (more detail discussed in Example 4). Enzymatic activity of inulosucrase was retained after the encapsulation.

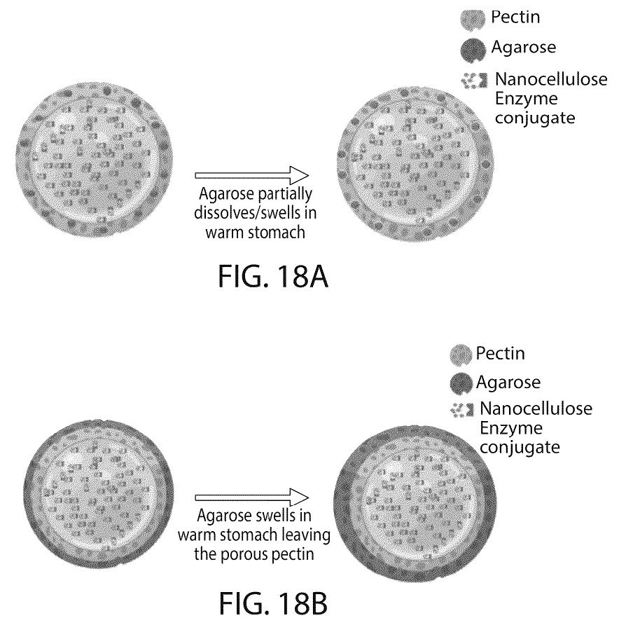

Alternative methods of encapsulation was also explored. For example, a pH and/or temperature-triggered particle (as described in Example 1) comprising agarose crosslinked with pectin shell could be used. For example, a microgel having an agarose crosslinked with pectin shell could be triggered to partially dissolve or swell in the stomach at a particular temperature (e.g., about 37° C.) to initiate enzymatic conversion of surrounding sugar (FIG. 18A). Downstream in the intestine, further degradation of the agarose-crosslinked-with-pectin shell in the higher pH environment would continue to increase enzyme and sugar interactions. The microgel could also have other morphologies. For instance, fluidic droplet could be encapsulated by an inner pectin shell and an outer agarose shell (FIG. 18B). For example, the double shell droplets may be prepared by using an initial aqueous solution of 4 wt % pectin as one layer and an aqueous solution of 1 wt % agarose as the outer layer. The double shell microcapsule exhibited enhanced barrier properties compared to single shell microcapsules.

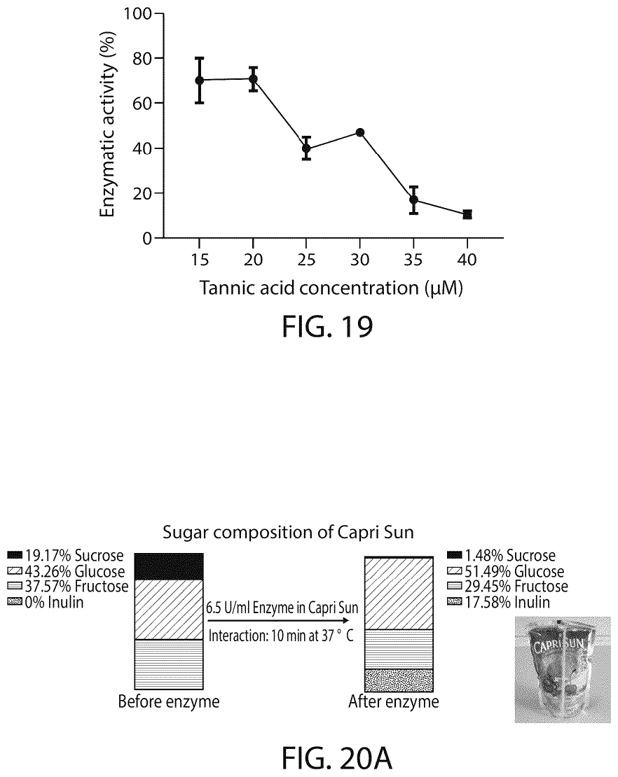

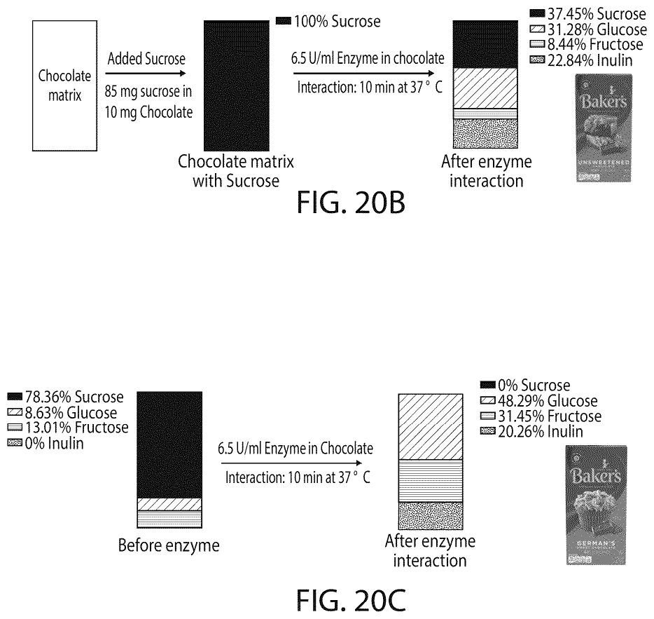

In addition to encapsulating the inulosucrase-nanocellulose complex, an inhibitor (e.g., tannic acid) could also be encapsulated together with the complex. The amount of tannic acid necessary to inhibit enzymatic activity of inulosucrase (while attached to nanocellulose) is shown in FIG. 19. As shown, increase in tannic acid concentration led to a reduction in enzymatic activity. It was demonstrated that inulosucrase activity could be inhibited by a polyphenol (e.g., tannic acid) and reactivated in the presence of bile salts. The effect of inulosucrase in various food products were studied. Free inulosucrase (not attached to any substrate) was added to a beverage (e.g., Capri San®) and the concentration of various saccharides was measured after 10 minutes at room temperature and 37° C. (FIG. 20A). As shown, inulosucrase led to significant reduction in sucrose level. Additional data for other food products (e.g., chocolates) are illustrated in FIGS. 20B-20C.

### Example 4

This Example describes a composition comprising fructosyltransferase enzymes entrapped in pectin microparticles, in accordance with some embodiments.

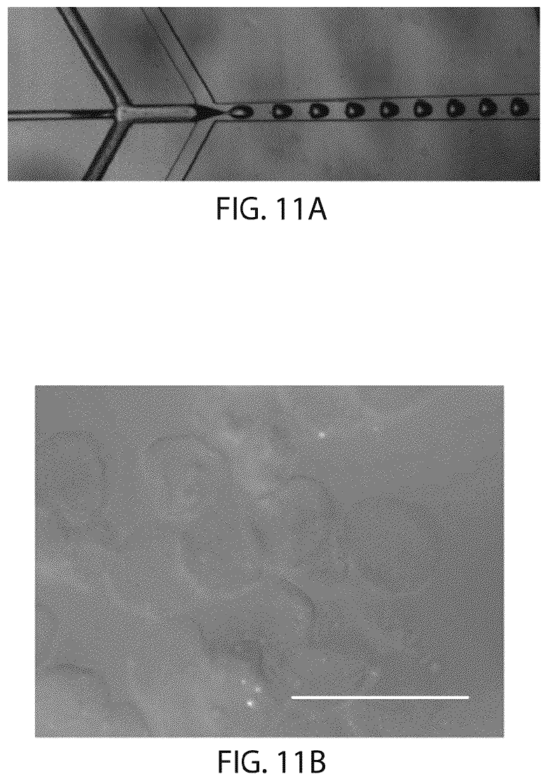

Fructosyltransferase enzymes (e.g., inulosucrase) were entrapped in pectin based microparticles and produced using microfluidics (FIG. 11A). The pectin based microparticle could contain 2 wt % of carboxymethyl cellulose. Particles were fabricated to have sizes between 80 to 100 microns (FIG. 11B).

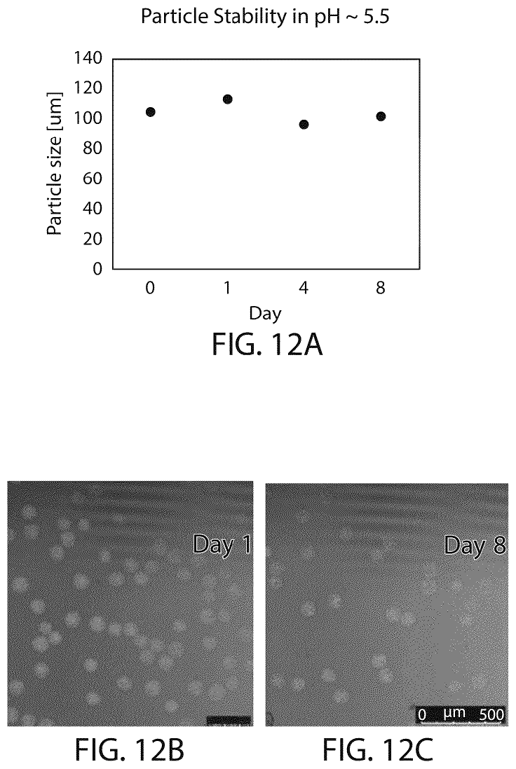

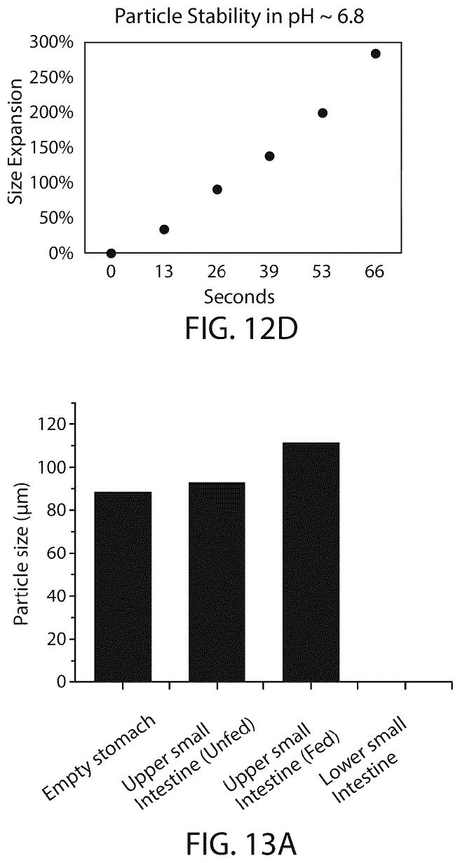

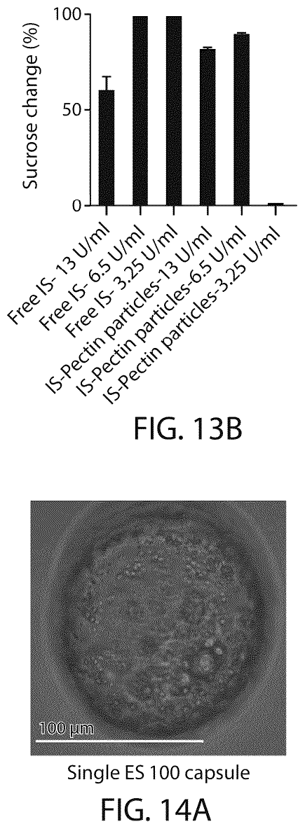

Particle stability was evaluated in aqueous buffers having different pH values. As shown in FIG. 12A-12B, particles stored in citric acid buffers (pH˜3.5) were stable up to 8 days (as suggested by the negligible change in particle size in FIG. 12A). Particle stability was also evaluated in other fluids having pH conditions that simulated various locations of the GI tract, e.g., simulated gastric fluid, simulated intestinal fluid, etc. (FIG. 13A). As shown by the change in particle size, pectin microparticles were stable in low pH buffers that simulated gastric fluid, and were unstable at higher pH buffers (e.g., pH of 6.8) that simulated lower intestinal fluid.

The effect of inulosucrase (IS) on sucrose reduction (%) for pectin particles with entrapped inulosucrase (IS-pectin particles) was evaluated (FIG. 13B). As shown, a significant change in sucrose was observed when the enzyme concentration in the particle is greater than 3.25 U/mL.

### Example 5

This Example describes a pH responsive microcapsule or double emulsions that can be used in a composition, in accordance with some embodiments.

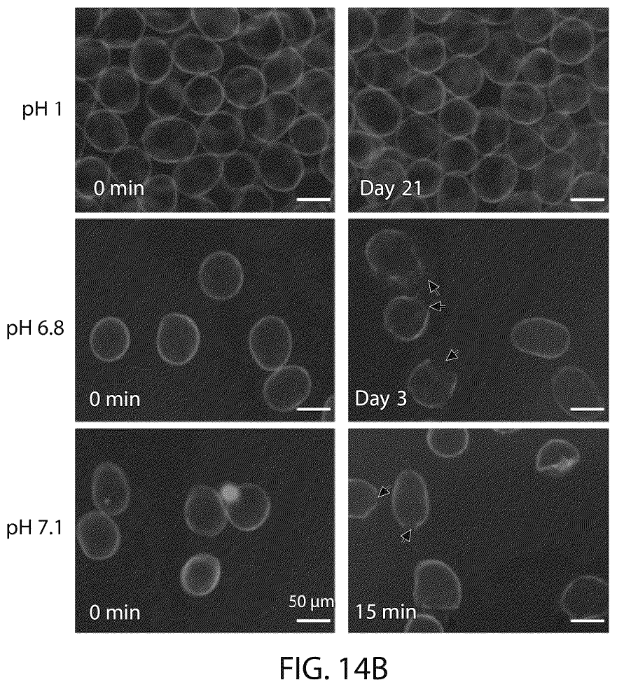

Microcapsules comprising pH sensitive polymers could be used to encapsulate various ingredients (e.g., enzymes) and achieve pH-controlled release of ingredients, i.e., such as gastrointestinal targeting in the ileum or colon. In this example, double emulsions were used as templates to form such microcapsules. The double emulsions comprised an inner phase comprising 3 wt % PVA, a middle phase comprising a commercially available polymer solution (e.g., Eudragit® or ES100 solution), and an outer phase comprising 5% PVA. The resulting ES100 microcapsule is shown in FIG. 14A. ES100 microcapsules was observed to be stable in low pH conditions (pH˜1) and unstable at neutral pH (pH˜6.8 to 7.1) (FIG. 14B). ES100 microcapsules could be used to encapsulate enzymes, to protect enzymes from glucose in gastric juice, and release enzymes in the intestinal juice. As an example, glucose oxidase was encapsulated in the ES100 microcapsules.

### Example 6

This Example describes pH responsive alginate based microcapsules and/or emulsions that can be used in a composition, in accordance with some embodiments.

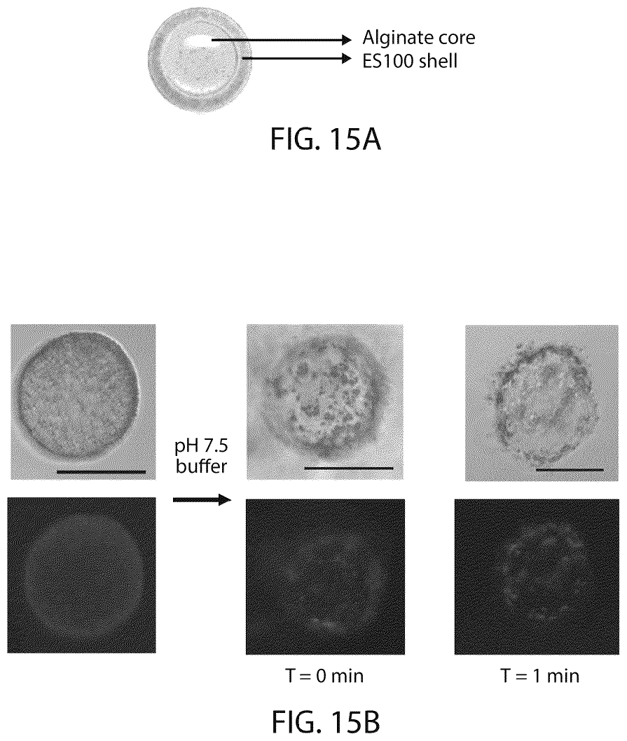

Microcapsules comprising an alginate core and an ES100 shell were formed from double emulsions produced using microfluidics (FIG. 15A). The microcapsules exhibited pH-dependent stability. The microcapsules were stable (e.g., remained intact) when exposed to a buffer having a pH of 1, but degraded in a buffer having a neutral pH (pH˜7.5, as shown in FIG. 15B).

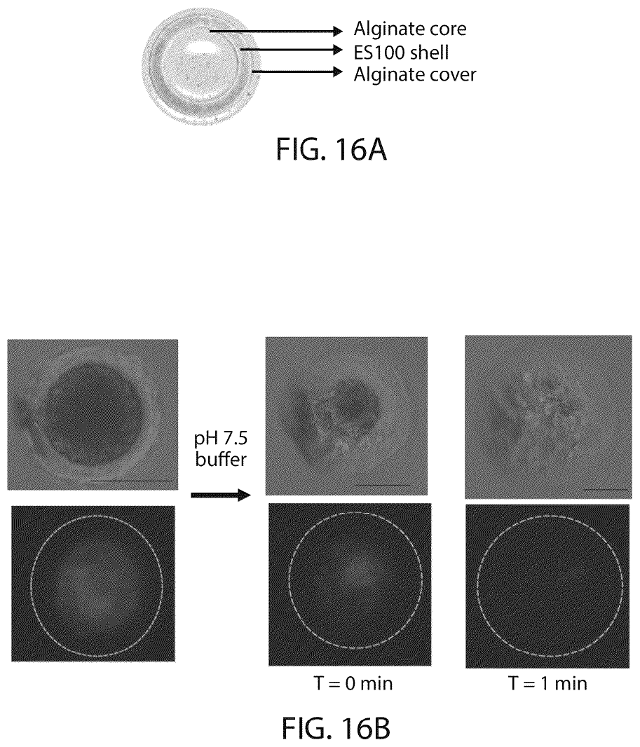

Microcapsules comprising an alginate core, an ES100 inner shell, and an alginate shell were formed from multiple emulsions (e.g., triple emulsion) produced using microfluidics (FIG. 16A). The microcapsules exhibited pH-dependent stability. As shown in FIG. 16B, the microcapsules were stable (e.g., remained intact) when exposed to a buffer having a pH of 1, but degraded in a buffer having a neutral pH (pH-7.5).

### Example 7

This Example describes polymeric particles containing enzymes conjugated to nanocellulose and use thereof for sugar reduction in intestine chips, in accordance with some embodiments.

A schematic showing the formation of particles containing nanocellulose-enzyme conjugates is illustrated in FIG. 21. The enzyme may be an enzyme (e.g., a fructosyltransferase enzyme) capable of converting fructose into a dietary fiber such as inulin. The enzyme may be conjugated with nanocellulose, thereby forming a nanocellulose-enzyme complex (NC-Enz) (e.g., as shown by complex 115 in FIG. 21). The conjugated nanocellulose-enzyme complex (NC-Enz) may offer several advantages compared to free enzymes, including: i) more stable enzymes, ii) better shelf life, iii) better retention of the enzymes in particles, and iv) added health benefits associated with nanocellulose.

The nanocellulose-enzyme complex may be further complexed with polyphenols (e.g., as shown by complex 120 in FIG. 21), which may provide inhibition and control of the enzymatic activity. The nanocellulose-enzyme conjugate complexed with polyphenol may be further contained within a natural polymer based particle (e.g., as shown by particle 130 in FIG. 21), e.g., such as a pectin particle. The natural polymer particles may protect enzymes from the surrounding and may comprise a pH and/or temperature responsive material, thereby allowing for controlled release of its inner cargo. Specifics regarding enzyme conjugation and particle formation are described in more detail below in the section on Experimental Protocols. Various sugar reduction experiments were performed using nanocellulose-enzyme (NC-Enz) conjugates, complex of NC-Enz with polyphenol, enzyme complexed with polyphenol, particle containing enzymes, etc., as illustrated in more detail below.

### I. Results

### Effect of Pectin Encapsulation

The sugar content in food products was measured when the food product was either exposed to pectin particles containing encapsulated enzymes (e.g., inulosucrase) or exposed to non-encapsulated free enzymes. To form pectin encapsulated enzyme particles, native enzyme (6.5 U/ml) was encapsulated in the pectin particles. The pectin particles had an average size of less than 100 microns. The particles containing the enzymes were then introduced in 0.5 mL of the food sample (Sucrose, 0.5 M) of pH 5.5 for 10 minutes at 37° C. While 92% of sugars in the food products was preserved when the food product was exposed to pectin encapsulated enzymes, only 37% of sugars was preserved when the food product was exposed to non-encapsulated enzymes. This indicates that pectin particles can be used to protect the sugar in food products from being degraded by the enzyme during shelf storage.

As shown in FIG. 12A, pectin particles encapsulated with native enzyme were stable for at least eight days at room temperature at pH 5.5. Size of the particles remained constant for over eight days. The pectin particles were also observed to demonstrated pH sensitivity, e.g., the particles were able to expand rapidly and release the encapsulated enzymes when exposed to a buffer having a pH of 6.8 or more (as shown in FIG. 12D).

### Effect of Nanocellulose-Enzyme Conjugates

The effect of free enzymes (e.g., inulosucrase) and nanocellulose-enzyme conjugates on sugar reduction were measured in various food products, e.g., such as Capri Sun®, unsweetened chocolate, and sweetened chocolate (FIGS. 20A-20C). The food products were observed to demonstrate 90% to 100% reduction in sucrose when exposed to free enzymes. Without wishing to be bound by theory, the presence of nanocellulose-enzyme conjugates may result in longer retention of the enzyme at a location (e.g., gut) in the subject during delivery (e.g., delivery to the gut), and thereby result in more persistent enzymatic activity and more efficient sugar reduction.

### Intestine Chip Experiments

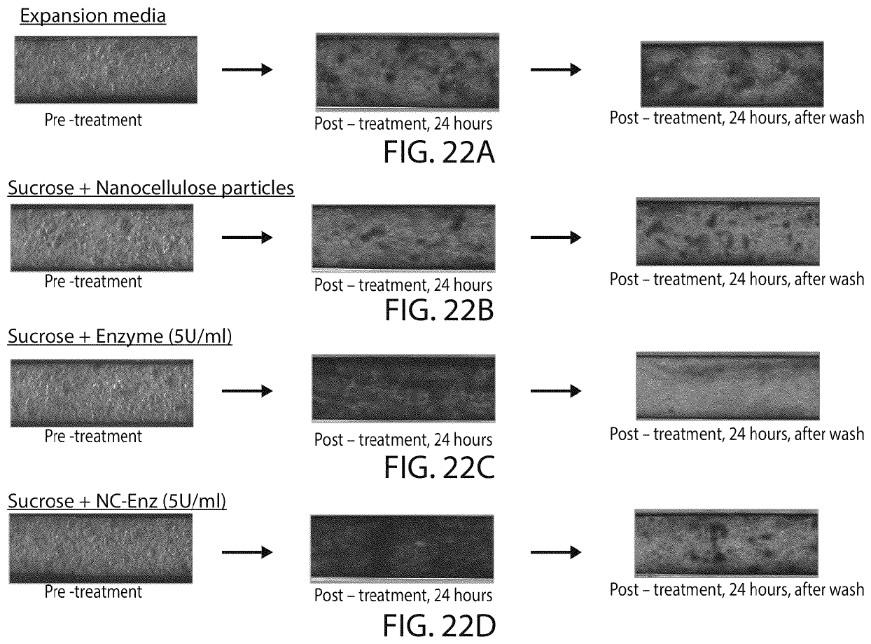

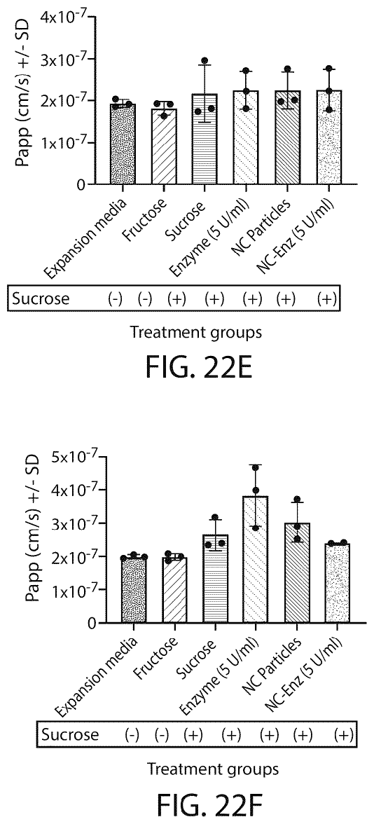

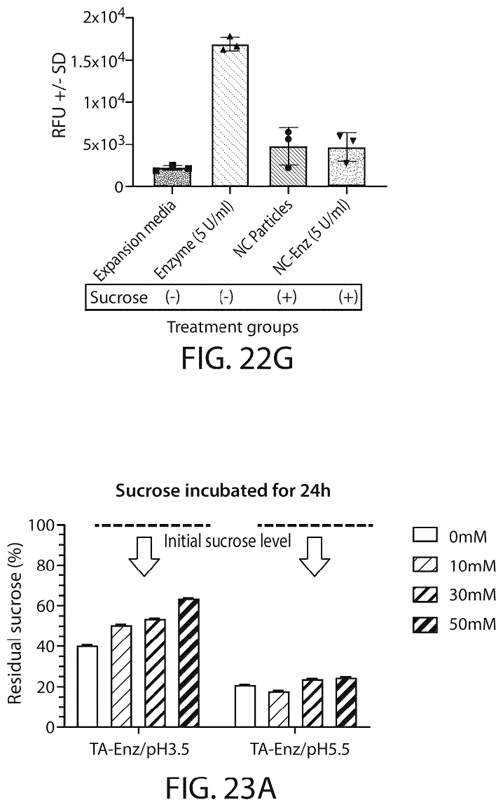

Intestine Chip Experiments were carried out to test the effect of free enzymes (non-conjugated enzymes) and nanocellulose-enzyme conjugates on sugar (e.g., sucrose) reduction and the formation of inulin. Experiments were performed using protocols described in the section below. FIGS. 22A-22D are representative images of cells on the apical channel of primary human small Intestine chips. Pre-treatment and post-treatment were taken at the same time points before and after particles and enzymes were introduced to the chips. The 3D structures on the image represent the Villi generated on the epithelial cells. As shown, inulin was produced and trapped in the mucus, which increased darkness on the post-treatment (after 24 hours) chips containing the enzyme or enzyme conjugates. Additionally, as shown in FIGS. 22C-22D, nanocellulose-enzyme conjugates demonstrated similar to slightly higher inulin formation compared to the free enzymes. Furthermore, more inulin were trapped in the mucus as a result of the scaffold created by the nanocellulose-enzyme conjugate present on the mucus.

Transepithelial barrier assessment of the primary cells (FIGS. 22E-22F) and Alamar blue assay (FIG. 22G) of primary cells on the intestine chip were performed according to the protocols described below. It was observed that nanocellulose and nanocellulose-enzyme conjugates were not toxic to the cells. No degradation of physical and structural properties of the epithelial cells was observed for 24 hours.

### Effect of Polyphenol on Enzymatic Activities

The effect of tannic acid on enzymatic activity in various food products at different pH conditions was studied. Tannic acid was combined with an enzyme (e.g., inulosucrase) at 1:1 molar ratio to form tannic acid-enzyme (TA-Enz) complex. The TA-Enz complex was prepared at various pH levels (e.g., 3.5, 5.5) and tannic acid concentrations (e.g., 10 mM, 30 mM, 50 mM). The effect of TA-Enz complex in sucrose solution and apple juice was compared to that of free enzyme (enzyme not complexed with tannic acid). Residual sucrose of sucrose solution and apple juice was first monitored in the presence of TA-Enz complexes after an incubation time of 15 minutes. All samples containing TA-Enz showed higher sucrose retention compared to the sample containing the free enzyme.

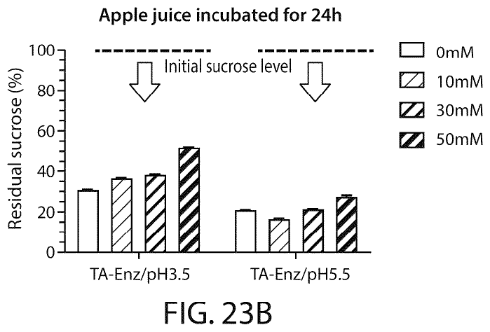

Residual sucrose of sucrose solution and apple juice was also monitored in the presence of TA-Enz complexes after an incubation time of 24 hours (FIGS. 23A-23B). All samples containing either the free enzymes (e.g., the 0 mM sample) or TA-Enz showed further sucrose reduction after 24 hours incubation. The TA-Enz complexes prepared at lower pH exhibited better enzyme inhibition effect compared to those prepared at higher pH. It was also observed that tannic acid can allow for repeated inhibition of enzymatic activity at low pH (e.g., pH˜3.5) and reactivation of enzymatic activity at higher pH (e.g., pH˜6.8).

### II. Experimental Protocols:

### Nanocellulose Enzyme Conjugation:

The enzyme attachment chemistry chosen was a carbodiimide conjugation, which included first activating carboxymethyl cellulose (CMC) carboxyl groups for direct reaction with primary amines that can be found on fructosyltransferase enzymes to form amide bonds. Activated CMC molecules were then adsorbed onto the surface of the nanocellulose (NC) particles. Finally, enzymes were attached to the amine groups created on the CMC-NC particles.

Specifically, 200 μL of 2.2 mg/mL calcium chloride (CaCl₂)) and 1 ml 0.2 g/l Carboxymethyl cellulose are in added in 0.8 ml MilliQ water. The solution was then mixed with 2 ml of 19.17 mg/ml 1-Ethyl-3-(3-dimethylaminopropyl)carbodiimide (EDC) and 46 mg/ml N-Hydroxysuccinimide (NHS) in 100 mM MES buffer. After 30 minutes at room temperature (RT), 100 mg NC was added to the solution. After 30 minutes of shaking, CMC adsorbed Nanocellulose was washed (2X) by the 50 mM MES buffer and once with 100 mM pH 5.5 acetate buffer. The CMC-NC particles were then incubated in a shaker with the known concentration of enzyme (6.5 U/ml) at room temperature for 30 minutes. The resulting mixture was centrifuged and washed with 100 mM pH 5.5 Acetate buffer. The concentration of the enzyme on the resulting conjugated NC-Enzyme solution was 2.2 U/ml. Finally, 10 mg NC-enzyme conjugate particles were aliquoted and used in the experiments.

### Enzyme Encapsulated Pectin Particles Formation

A microfluidic drop generation method was used to generate pectin particles. Enzymes, 0.5% CMC, and CaCO₃ were mixed in 2% pectin solution. The water in oil particles were formed using a microfluidic device, with pectin solution as the water phase and corn oil with 0.02% acetic acid as the continuous phase.

### Sugar Reduction Experiments

6.5 U/ml native enzyme, or 10 mg nanocellulose-enzyme (2.2 U/ml) conjugates, or pectin particles with equivalent concentration of encapsulated enzyme, were mixed with 0.5 ml sugar substrates (0.1 M sucrose solution, juice, chocolate solution). The mixture is incubated at 37° C. for 10 minutes and at 100° C. for 10 minutes. At 100° C. the enzymes were deactivated. The solution was then used for different assays, such as inulin, glucose, fructose assays.

### Inulin, Glucose/Fructose Assay

A commercial Fructan kit from Megazyme was used to detect FOS (inulin) produced from the enzyme and sugar interactions. A sucrose/D-glucose/D-Fructose assay was performed to detect and analyze the sugar content in the samples. Sucrose/glucose/fructose detection was performed using HPLC with RI/CAD and HILIC column.

### Intestine Chip Experiments

Intestine Chips were fabricated from PDMS and were obtained from Emulate Inc. Chips were activated by oxygen plasma treatment for 1.5 minutes and followed by: i) incubation with (3-Aminopropyl)triethoxysilane (2% vol/vol in ethyl alcohol) for 30 minutes at room temperature, ii) washing in ethyl alcohol, and iii) incubating the chips at 80° C. overnight. Type I collagen (200 μg/mL) and Matrigel (1% in PBS) were then introduced into channels in the chip. The chips were incubated in a humidified incubator at 37° C. for 2 hours before being washed with PBS. Epithelial cells were then resuspended in expansion media (EM) (6×10⁶ cells/ml; of which 30 μL was used to seed each chip and thus resulting in ˜180,000 cells/chip), infused into the top channel, and incubated overnight in static at 37° C. The chip was perfused with EM (pH 6.0) at 60 μL/h through top and bottom channels. At day 14, transepithelial barrier assessment assay was performed on the chips (baseline measurement) and imaged at least one chip per treatment group (FIG. 23E). The chips were then treated with different solution conditions (FIGS. 22A-22D). Samples were collected at 2 hours and 24 hours after treatment. Finally, after 24 hours post treatment, Alamar blue assay was performed for viability and toxicity assessment of the cells in the chips (FIG. 23F).

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition, comprising:
a particle comprising:
(a) an enzyme capable of polymerizing a sugar to produce a polymer; and
(b) an inhibitor associated with the enzyme, wherein the inhibitor inhibits the enzyme from polymerizing the sugar, and wherein the inhibitor is dissociated from the enzyme when the particle is exposed to an ionic strength of at least 5 mmol/L and a pH of greater than 3.5.

2. The composition of claim 1, wherein the ionic strength is at least 10 mmol/L.

3. The composition of any one of claims 1-2, wherein the ionic strength is at least 25 mmol/L.

4. The composition of any one of claims 1-3, wherein the ionic strength is at least 50 mmol/L.

5. The composition of any one of claims 1-4, wherein the enzyme is attached to a substrate.

6. The composition of claim 5, wherein the substrate comprises nanocellulose.

7. The composition of any one of claims 5-6, wherein the enzyme is attached to the substrate via a polymer.

8. The composition of claim 7, wherein the polymer comprises carboxymethyl cellulose.

9. The composition of any one of claims 1-8, wherein the inhibitor comprises a polyphenol.

10. The composition of claim 9, wherein the polyphenol comprises a polyphenol selected from the group consisting of tannic acid, gallic acid and Epigallocatechi gallate (EGCG).

11. The composition of any one of claims 1-10, wherein a binding affinity between the inhibitor and the enzyme is affected by a change in pH and/or ionic strength in the composition.

12. The composition of any one of claims 1-11, wherein the inhibitor is present in amount of greater than or equal to 1 micromolar and less than or equal to 100 mM.

13. The composition of any one of claims 1-12, wherein the inhibitor is present in amount of greater than or equal to 50 micromolar and less than or equal to 100 micromolar.

14. The composition of any one of claims 1-13, further comprising an additive.

15. The composition of claim 14, wherein the additive comprises a mineral.

16. The composition of claim 15, wherein the mineral is present in an amount of greater than or equal to 5 mM in the composition.

17. The composition of any one of claims 15-16, wherein the mineral comprises a mineral selected the group consisting of CaCl₂), FeCl₃, MgCl₂, and ZnCl₂.

18. The composition of any one of claims 14-17, wherein the additive comprises a bile salt.

19. The composition of claim 18, wherein the bile salt is present in an amount of greater than or equal to 0.2 wt % of a total weight of the composition.

20. The composition of any one of claims 14-19, wherein the additive comprises a metal ion.

21. The composition of claim 20, wherein the metal ion is a transition metal ion and/or an alkaline-earth metal ion.

22. The composition of any one of claims 20-21, wherein the metal ion comprises a metal ion selected from the group consisting of iron (III), zinc (II), calcium, magnesium, and aluminum.

23. The composition of any one of claims 20-22, wherein the metal ion comprises a metal ion selected from the group consisting of calcium and magnesium.

24. The composition of any one of claims 20-23, wherein the metal ion is a cation having an oxidation state of at least +2.

25. The composition of any one of claims 20-24, wherein a binding affinity between the inhibitor and the metal ion is affected by a change in pH and/or temperature in the composition.

26. The composition of any one of claims 20-25, wherein when the composition is exposed to a relatively low pH, a binding affinity between the enzyme and the inhibitor is greater than a binding affinity between the inhibitor and the metal ion.

27. The composition of any one of claims 20-26, wherein when the composition is exposed to a relatively high pH, a binding affinity between the enzyme and the inhibitor is less than a binding affinity between the inhibitor and the metal ion.

28. The composition of any one of claims 20-27, wherein the metal ion is present in an amount of greater than or equal to 5 mmol/L in the composition.

29. The composition of any one of claims 20-28, wherein the metal ion is present in an amount of greater than or equal to 10 mmol/L in the composition.

30. The composition of any one of claims 20-29, wherein the metal ion is present in an amount of greater than or equal to 25 mmol/L in the composition.

31. The composition of any one of claims 20-30, wherein the metal ion is present in an amount of greater than or equal to 50 mmol/L in the composition.

32. The composition of any one of claims 1-31, wherein the enzyme is selected from the group consisting of glycosyltransferase, fructosyltransferase, hexosyltransferase, sucrase, amylosucrase, glucansucrase, levansucrase, inulosucrase, sucrose:fructose fructosyltransferase, and fructose:fructose fructosyltransferase.

33. The composition of any one of claims 1-32, wherein the enzyme is chemically or physically attached to a substrate and contained within the particle.

34. The composition of any one of claims 1-33, wherein the enzyme is contained within the particle.

35. The composition of any one of claims 1-34, wherein the particle is a fluidic droplet, a double emulsion, a microcapsule, and/or a polymeric particle.

36. The composition of any one of claims 1-35, wherein the particle comprises one or more of nanocellulose, nanocrystal cellulose, nanofiber cellulose, cellulose derivatives, polyvinyl alcohol, alginate, agarose, pectin, glucomannan, phytagel, pullulan, inulin, xanthan gum.

37. The composition of any one of claims 1-36, wherein the particle comprises a fluidic droplet.

38. The composition of claim 37, wherein the fluidic droplet forms part of an emulsion.

39. The composition of any one of claims 37-38, wherein the fluidic droplet is contained with an immiscible carrying fluid.

40. The composition of any one of claims 37-39, wherein the particle comprises a shell surrounding the fluidic droplet configured to prevent fluidic communication between the enzyme and a sugar.

41. The composition of claim 40, wherein the shell comprises a polymer.

42. The composition of any one of claims 40-41, wherein the shell comprises agarose and/or pectin.

43. The composition of any one of claims 40-42, wherein the shell comprises a first shell comprising agarose and a second shell comprising pectin.

44. The composition of any one of claims 40-42, wherein the shell comprises agarose crosslinked with pectin.

45. The composition of any one of claims 40-44, wherein the shell dissociates and/or swells when exposed to a change in pH and/or temperature, such that the enzyme is in direct contact with a sugar.

46. The composition of any one of claims 40-45, wherein the enzyme is in direct contact with the sugar when the shell is exposed to a temperature of at least 35° C.

47. The composition of any one of claims 1-46, wherein the sugar comprises a molecule selected from the group consisting of glucose, sucrose, fructose, lactose, galactose, and maltose.

48. The composition of any one of claims 1-47, wherein the polymer comprises a polymer selected from the group consisting of oligosaccharides, fructo-oligosaccharides, gluco-oligosaccharides, malto-oligosacchrides, amylose, levan, and inulin.

49. The composition of any one of claims 1-48, wherein the polymer comprises a prebiotic.

50. The composition of any one of claims 1-49, further comprising a supplement comprising a probiotic.

51. A method of using the composition of any one of claims 1-50, comprising swallowing the composition.

52. A method of making the composition of any one of claims 1-51, comprising:
combining the enzyme and the inhibitor to form a complex; and
associating the complex with the particle.

53. The method of claim 52, wherein associating the complex with a particle comprises encapsulating and/or attaching the complex to the particle.

54. A composition, comprising:
a particle comprising:
(a) an enzyme capable of polymerizing a monosaccharide or a disaccharide to produce a polysaccharide; and
(b) an inhibitor associated with the enzyme, wherein the inhibitor inhibits the enzyme from polymerizing the monosaccharide or disaccharide.

55. The composition of claim 54, wherein the monosaccharide or the disaccharide comprises a molecule selected from the group consisting of glucose, sucrose, fructose, lactose, galactose, and maltose.

56. The composition of any one of claims 54-55, wherein the polysaccharide comprises a molecule selected from the group consisting of oligosaccharides, fructo-oligosaccharides, gluco-oligosaccharides, malto-oligosacchrides, amylose, levan, and inulin.

57. The composition of any one of claims 54-56, wherein the inhibitor comprises a polyphenol.

58. The composition of any one of claims 54-57, wherein the enzyme comprises a glycosyltransferase.

59. The composition of claim 58, wherein the glycosyltransferase comprises an enzyme selected from the group consisting of fructosyltransferase, sucrose:fructose fructosyltransferase, fructose:fructose fructosyltransferase, hexosyltransferase, sucrase, amylosucrase, glucansucrase, levansucrase, and inulosucrase.

60. The composition of any one of claims 54-59, wherein the enzyme is attached to a substrate.

61. The composition of claim 60, wherein the substrate comprises nanocellulose.

62. The composition of any one of claims 60-61, wherein the enzyme is attached to the substrate via a polymer.

63. The composition of claim 62, wherein the polymer comprises carboxymethyl cellulose.

64. The composition of any one of claims 54-63, further comprising an additive.

65. The composition of claim 64, wherein the additive comprises a mineral.

66. The composition of any one of claims 64-65, wherein the additive comprises a metal ion.

67. The composition of claim 66, wherein the metal ion is a transition metal ion and/or an alkaline-earth metal ion.

68. A composition, comprising:
a particle, comprising:
(a) one or more enzymes, wherein the one or more enzymes comprises an enzyme selected from the group consisting of glucose oxidase, glucose dehydrogenase, glycosyltransferase, fructosyltransferase, hexosyltransferase, sucrase, amylosucrase, glucansucrase, levansucrase, inulosucrase, fructose dehydrogenase, glucose-fructose oxidoreductase, beta-galactosidase, amylase, cellulase, and laccase; and
(b) a polyphenol associated with the one or more enzymes.

69. The composition of claim 68, wherein the one or more enzymes comprises a glucose oxidase capable of converting a glucose into gluconic acid.

70. The composition of any one of claims 68-69, wherein the one or more enzymes are attached to a substrate.

71. The composition of claim 70, wherein the substrate comprises nanocellulose.

72. The composition of any one of claims 70-71, wherein the one or more enzymes are attached to the substrate via a polymer.

73. The composition of claim 72, wherein the polymer comprises carboxymethyl cellulose.

74. The composition of any one of claims 68-73, wherein the polyphenol is selected from the group consisting of tannic acid, chlorogenic acid, quercetin, and Epigallocatechi gallate (EGCG).

75. The composition of any one of claims 68-74, wherein polyphenol is present in amount of greater than or equal to 1 μM.

76. The composition of any one of claims 68-75, further comprising an additive.

77. The composition of claim 76, wherein the additive comprises a mineral.

78. The composition of any one of claims 76-77, wherein the additive comprises a metal ion.

79. The composition of claim 78, wherein the metal ion is a transition metal ion and/or an alkaline-earth metal ion.

80. A composition, comprising:
digestive juice comprising a particle, the particle comprising:
(a) an enzyme capable of polymerizing a sugar to produce a polymer; and
(b) an inhibitor capable of inhibiting the enzyme from polymerizing the sugar.

81. The composition of claim 80, wherein the digestive juice comprises intestinal juice.

82. The composition of any one of claims 80-81, wherein the digestive juice comprises gastric juice.

83. The composition of any one of claims 80-82, wherein the digestive juice comprises bile salt.

84. The composition of any one of claims 80-83, wherein the sugar comprises a monosaccharide and/or disaccharide selected from the group consisting of glucose, sucrose, fructose, lactose, galactose, and maltose.

85. The composition of any one of claims 80-84, wherein the polymer comprises a polymer selected from the group consisting of oligosaccharides, fructo-oligosaccharides, malto-oligosacchrides, gluco-oligosaccharides, amylose, levan, and inulin.

86. The composition of any one of claims 80-85, wherein the inhibitor comprises a polyphenol.

87. The composition of any one of claims 80-86, wherein the enzyme comprises an enzyme selected from the group consisting of glycosyltransferase, fructosyltransferase, sucrose:fructose fructosyltransferase, fructose:fructose fructosyltransferase, hexosyltransferase, sucrase, amylosucrase, glucansucrase, levansucrase, and inulosucrase.

88. The composition of any one of claims 80-87, wherein the enzyme is attached to a substrate.

89. The composition of claim 88, wherein the substrate comprises nanocellulose.

90. The composition of any one of claims 88-89, wherein the enzyme is attached to the substrate via a polymer.

91. The composition of claim 90, wherein the polymer comprises carboxymethyl cellulose.

92. The composition of any one of claims 80-91, further comprising an additive.

93. The composition of claim 92, wherein the additive comprises a mineral.

94. The composition of any one of claims 92-93, wherein the additive comprises a metal ion.

95. A composition, comprising:
a particle comprising:
(a) an enzyme capable of converting a sugar from an original form into a form that is non-digestible or less digestible compared to the original form; and
(b) an inhibitor that inhibits the enzyme from converting the sugar, wherein upon a change in a condition associated with the composition, the enzyme converts the sugar into the non-digestible or less digestible form, and wherein the condition comprises pH, temperature, and/or ionic strength.

96. The composition of claim 95, wherein the non-digestible or less digestible form of the sugar comprises one or more of oligosaccharides, fructo-oligosaccharides, gluco-oligosaccharides, malto-oligosacchrides, amylose, levan, and inulin.

97. The composition of any one of claims 95-96, wherein the condition comprises a pH of greater than 3.5.

98. The composition of any one of claims 95-97, wherein the condition comprises a temperature of at least 35° C.

99. The composition of any one of claims 95-98, wherein the conditions comprises an ionic strength of at least 50 mmol/L.

100. The composition of any one of claims 95-99, wherein the one or more enzymes comprises a glucose oxidase capable of converting a glucose into gluconic acid.

101. The composition of any one of claims 95-100, wherein the inhibitor comprises a polyphenol.

102. The composition of any one of claims 95-101, further comprising an additive.

103. The composition of claim 102, wherein the additive comprises a mineral.

104. The composition of any one of claims 102-103, wherein the additive comprises a metal ion.

105. The composition of claim 104, wherein the metal ion is a transition metal ion and/or an alkaline-earth metal ion.

106. The composition of any one of claims 95-105, wherein the enzyme is attached to a substrate.

107. The composition of claim 106, wherein the substrate comprises nanocellulose.

108. A composition, comprising:
a particle, comprising:
(a) one or more enzymes, wherein the one or more enzymes comprises an enzyme selected from the group consisting of glucose oxidase, glucose dehydrogenase, glycosyltransferase, fructosyltransferase, hexosyltransferase, sucrase, amylosucrase, glucansucrase, levansucrase, inulosucrase, fructose dehydrogenase, glucose-fructose oxidoreductase, beta-galactosidase, amylase, cellulase, laccase;
(b) an inhibitor that inhibits the enzyme from converting the sugar.

109. The composition of claim 108, wherein the one or more enzymes comprises a glucose oxidase capable of converting a glucose into gluconic acid.

110. The composition of any one of claims 108-109, wherein the inhibitor comprises a polyphenol.

111. The composition of any one of claims 108-110, wherein the enzyme is attached to a substrate.

112. The composition of claim 111, wherein the substrate comprises nanocellulose.

113. The composition of any one of claims 111-112, wherein the enzyme is attached to the substrate via a polymer.

114. The composition of claim 113, wherein the polymer comprises carboxymethyl cellulose.

115. The composition of any one of claims 108-114, further comprising an additive.

116. The composition of claim 115, wherein the additive comprises a mineral.

117. The composition of any one of claims 115-116, wherein the additive comprises a metal ion.

118. The composition of claim 117, wherein the metal ion is a transition metal ion and/or an alkaline-earth metal ion.

119. A composition, comprising:
a particle comprising an enzyme capable of polymerizing a sugar to produce a polymer.

120. The composition of claim 119, wherein the enzyme is attached to a substrate.

121. The composition of claim 120, wherein the substrate comprises nanocellulose.

122. The composition of any one of claims 120-121, wherein the enzyme is attached to the substrate via a polymer.

123. The composition of claim 122, wherein the polymer comprises carboxymethyl cellulose.

124. The composition of any one of claims 119-123, wherein the enzyme is selected from the group consisting of glycosyltransferase, fructosyltransferase, hexosyltransferase, amylosucrase, glucansucrase, sucrase, levansucrase, inulosucrase, sucrose:fructose fructosyltransferase, and fructose:fructose fructosyltransferase.

125. The composition of any one of claims 119-124, wherein the particle is a fluidic droplet, a double emulsion, a microcapsule, and/or a polymeric particle.

126. The composition of any one of claims 119-125, wherein the sugar comprises a molecule selected from the group consisting of glucose, sucrose, fructose, lactose, galactose, and maltose.

127. The composition of any one of claims 119-126, wherein the polymer comprises a polymer selected from the group consisting of oligosaccharides, fructo-oligosaccharides, malto-oligosacchrides, gluco-oligosaccharides, levan, and inulin.

128. The composition of any one of claims 119-127, wherein the particle further comprises an inhibitor associated with the enzyme, wherein the inhibitor inhibits the enzyme from polymerizing the sugar.

129. The composition of claim 128, wherein the inhibitor comprises a polyphenol.

130. The composition of any one of claims 119-129, wherein the particle further comprises an additive.

131. The composition of claim 130, wherein the additive comprises a mineral.

132. The composition of any one of claims 130-131, wherein the additive comprises a metal ion.

133. A composition, comprising:
a particle comprising one or more enzymes, wherein the one or more enzymes comprises an enzyme selected from the group consisting of glucose oxidase, glucose dehydrogenase, glycosyltransferase, fructosyltransferase, hexosyltransferase, amylosucrase, glucansucrase, sucrase, levansucrase, inulosucrase, fructose dehydrogenase, glucose-fructose oxidoreductase, beta-galactosidase, amylase, cellulase, laccase.

134. The composition of claim 133, wherein the one or more enzymes comprises a glucose oxidase capable of converting a glucose into gluconic acid.

135. The composition of any one of claims 133-134, wherein the particle further comprises a polyphenol associated with the one or more enzymes.

136. The composition or method of any one of claims 1-135, further comprising a primer comprising inulin and/or amylose.

137. The composition or method of any one of claims 1-136, wherein the particle comprises a material selected from the group of pectin, inulin, amylose, agarose, alginate, gelatin, phytogel, glucomennan, and/or nanocellulose fiber.

138. The composition of method of any one of claims 1-137, wherein the particle comprises nanocellulose fiber.
